(19) United States
(12) Patent Application Publication
(10) Pub. No.: US 2026/0033714 A1
(43) Pub. Date: Feb. 5, 2026

(54) OPHTHALMIC APPARATUS, METHOD OF PROCESSING OPHTHALMIC IMAGE, AND METHOD OF CONTROLLING OPHTHALMIC APPARATUS

(71) Applicant: TOPCON CORPORATION, Tokyo (JP)

(72) Inventor: Shigeru HONDA, Tokyo (JP)

(73) Assignee: TOPCON CORPORATION, Tokyo (JP)

(21) Appl. No.: 19/283,325

(22) Filed: Jul. 29, 2025

(30) Foreign Application Priority Data
Jul. 31, 2024 (JP) 2024-124725

(51) Int. Cl. A61B 3/10; A61B 3/12; G06T 5/50; G06T 5/70

(52) U.S. Cl. CPC A61B 3/102; A61B 3/12; G06T 5/50; G06T 5/70; G06T 2207/10101; G06T 2207/30041

(57) ABSTRACT

An ophthalmic apparatus of an embodiment example includes an image acquisition unit, image projection processor, blood vessel enhancement processor, denoising processor, and image compositing processor. The image acquisition unit acquires an optical coherence tomography angiography image of a fundus of a subject's eye. The image projection processor applies a projection process to the optical coherence tomography angiography image to generate a projection image. The blood vessel enhancement processor applies a blood vessel enhancing filter that is configured to enhance a blood vessel image to the projection image to generate a blood vessel enhanced image. The denoising processor applies a denoising process to the projection image to generate a denoised image. The image compositing processor applies an image compositing process to the projection image, the blood vessel enhanced image, and the denoised image to generate a composite image.

### CROSS-REFERENCE TO RELATED APPLICATION

This application is based upon and claims the benefit of priority from Japanese Patent Application No. 2024-124725, filed Jul. 31, 2024; the entire contents of which are incorporated herein by reference.

### FIELD

The present disclosure relates to an ophthalmic apparatus, a method of processing an ophthalmic image, and a method of controlling an ophthalmic apparatus.

### BACKGROUND

Various types of imaging modalities are used in ophthalmic examination, one of which is optical coherence tomography (hereinafter referred to as OCT). OCT can be used not only for structural imaging but also for functional imaging, and is one of the modalities that has attracted the most attention in recent years.

One method of functional imaging using OCT is OCT angiography (hereinafter referred to as OCTA). OCTA is a functional imaging modality for depicting or visualizing blood flow, and is typically used to obtain eye fundus blood vessel images (retinal blood vessel images, choroidal blood vessel images, etc.). Such application is disclosed in U.S. Patent Application Publication No. 2022/0151568 and U.S. Pat. No. 10,136,812. Signals from fundus tissue (structure) do not change over time, whereas signals from blood flow inside blood vessels change over time. OCTA is an imaging technique that focuses on this fact, and constructs a blood vessel image by enhancing the area where there is a temporal signal change (blood flow signals). OCTA is also referred to as OCT motion contrast imaging. Images constructed by OCTA are referred to as OCTA images, OCT angiograms, motion contrast images, etc.

The techniques described in the above two documents use a multiscale Frangi filter to enhance blood vessels. Details of this filter are described in the following document: Alejandro F. Frangi, Wiro J. Niessen, Koen L. Vincken & Max A. Viergever. Multiscale vessel enhancement filtering. In International Conference on Medical Image Computing and Computer-Assisted Intervention-MICCAI'98: First International Conference, Cambridge, MA, USA, Oct. 11-13, 1998, Proceedings (Lecture Notes in Computer Science, 1496), pp. 130-137, Springer Berlin Heidelberg. Briefly, the Frangi filter is a filter for extracting and enhancing linear and tubular structures using two eigenvalues of a Hessian matrix whose elements (entries) are second-order derivatives in each direction of the image. The Frangi filter evaluates the likelihood of a given pixel being a blood vessel, that is, evaluates the probability that a given pixel corresponds to a blood vessel. Multiscale Frangi filtering is a method or technique that uses a plurality of different scales to extract structures of various dimensions.

A multiscale Frangi filter is used in various fields, and is widely used in the medical field to enhance blood vessel images and nerve images. However, the inventors of the present disclosure have gained a finding that there are cases in which some problems occur in images obtained by applying multiscale Frangi filters.

For example, in an image obtained by applying a multiscale Frangi filter to an angiographic image generated by OCTA, irregularity in brightness (luminance) may occur in an image of a relatively thick (large) blood vessel, dropout (absence) of an image of a relatively thin (small) blood vessel may occur, and vessel-like noise may occur in an image of a foveal avascular zone (hereinafter referred to as FAZ). The problem of the irregularity in brightness in the image of the thick blood vessel is that the brightness in the vicinity of the centerline (axis line) of the thick blood vessel is reduced, resulting in the region being depicted dark. The problem of the dropout of the image of the thin blood vessel is that the visibility of the thin blood vessel is relatively reduced due to the enhancement of thick blood vessels. The problem of the noise in the FAZ is that not only the image of the blood vessel but also the noise is enhanced, so that noise that looks like a blood vessel appears in the FAZ, where there should be no signal (therefore no image) corresponding to a blood vessel.

It has also been confirmed that similar problems may occur when using filters other than a multiscale Frangi filter. For example, similar problems may occur when using Gabor filters, non-local means filters, wavelet filters, or other filters.

### BRIEF SUMMARY

One non-limiting objective of some aspect examples of the present disclosure is to address problems caused by the application of an image filter used for blood vessel enhancement.

A non-limiting objective of some aspect examples of the present disclosure is to address at least one of the following problems: irregularity in brightness in an image of a thick blood vessel, dropout of an image of a thin blood vessel, and noise in FAZ.

An ophthalmic apparatus according to some aspect examples of the present disclosure includes an image acquisition unit, an image projection processor, a blood vessel enhancement processor, a denoising processor, and an image compositing processor. The image acquisition unit is configured to acquire an OCTA image of a fundus of a subject's eye. The image projection processor is configured to apply a projection process to the OCTA image to generate a projection image. The blood vessel enhancement processor is configured to apply a blood vessel enhancing filter that is configured to enhance a blood vessel image to the projection image to generate a blood vessel enhanced image. The denoising processor is configured to apply a denoising process to the projection image to generate a denoised image. The image compositing processor is configured to apply an image compositing process to the projection image, the blood vessel enhanced image, and the denoised image to generate a composite image.

### BRIEF DESCRIPTION OF THE SEVERAL VIEWS OF THE DRAWINGS

FIG. 1 is a schematic diagram of the configuration of an ophthalmic apparatus according to a non-limiting embodiment example.

FIG. 2 is a schematic diagram of the configuration of an ophthalmic apparatus according to a non-limiting embodiment example.

FIG. 3 is a schematic diagram of the configuration of an ophthalmic apparatus according to a non-limiting embodiment example.

FIG. 4 is a schematic diagram of the configuration of an ophthalmic apparatus according to a non-limiting embodiment example.

FIG. 5A is a flowchart of the operation of an ophthalmic apparatus according to a non-limiting embodiment example.

FIG. 5B is a schematic diagram of the operation of an ophthalmic apparatus according to a non-limiting embodiment example.

FIG. 6 is a comparative example in relation to the operation of an ophthalmic apparatus according to a non-limiting embodiment example.

FIG. 7 is a flowchart of the operation of an ophthalmic apparatus according to a non-limiting embodiment example.

FIG. 8A is a flowchart of the operation of an ophthalmic apparatus according to a non-limiting embodiment example.

FIG. 8B is a schematic diagram of the operation of an ophthalmic apparatus according to a non-limiting embodiment example.

FIG. 9 is a flowchart of the operation of an ophthalmic apparatus according to a non-limiting embodiment example.

FIG. 10A is a flowchart of the operation of an ophthalmic apparatus according to a non-limiting embodiment example.

FIG. 10B is a schematic diagram of the operation of an ophthalmic apparatus according to a non-limiting embodiment example.

FIG. 11A is a flowchart of the operation of an ophthalmic apparatus according to a non-limiting embodiment example.

FIG. 11B is a schematic diagram of the operation of an ophthalmic apparatus according to a non-limiting embodiment example.

FIG. 12 is a flowchart of the operation of an ophthalmic apparatus according to a non-limiting embodiment example.

### DETAILED DESCRIPTION

Several aspect examples of embodiment examples according to the present disclosure will be described. In the present disclosure, several aspect examples will be described for each of the following embodiment examples: embodiment examples of an ophthalmic apparatus (e.g., an ophthalmic imaging apparatus, an ophthalmic image processing apparatus, etc.), embodiment examples of a method of processing an ophthalmic image, embodiment examples of a method of controlling an ophthalmic apparatus, embodiment examples of a program, and embodiment examples of a recording medium. Each aspect example provides a non-limiting embodiment example.

Several embodiment examples according to the present disclosure can be adopted to address problems caused by image filters used for blood vessel enhancement. There are various problems caused by image filters used for blood vessel enhancement. Among these problems, the present disclosure particularly addresses three specific issues, which will be described in detail below. It should be noted that a person skilled in the art would understand that problems that can be addressed by the technique or technology according to the present disclosure are not limited to the three issues.

As a premise for understanding the problems that the present disclosure focuses on, a Frangi filter, which is a representative example of image filters used for blood vessel enhancement, will be described. The Frangi filter in the present disclosure is an image filter configured to detect vascular structures in an eye image using two eigenvalues of a Hessian matrix. In a multiscale Frangi filter, a plurality of different scales is used to extract blood vessels of various dimensions (various thicknesses).

The inventors of the present disclosure have obtained findings regarding various problems that arise as a result of examining various images acquired by applying multiscale Frangi filters to many OCTA images. In particular, it has been found that the three problems described below are relatively prominent in terms of occurrence frequency and degree.

The first problem that may arise in an image obtained by applying a multiscale Frangi filter to an OCTA image (hereinafter referred to as a blood vessel enhanced image) is that irregularity in brightness occurs in the images of relatively thick blood vessels among images of blood vessels of various thicknesses depicted in the blood vessel enhanced image. More specifically, the first problem is that the brightness in the vicinity of the centerline in the image of a relatively thick blood vessel is low, that is, the region near the centerline is depicted dark.

The second problem is the dropout of the images of relatively thin blood vessels that should be clearly depicted in the blood vessel enhanced image. The second problem is considered to be due to the fact that the multiscale Frangi filter enhances the images of relatively thick blood vessels, which relatively reduces the visibility of the images of relatively thin blood vessels.

The third problem is that, in a blood vessel enhanced image of an area including an avascular region of the fundus (e.g., FAZ), noise that looks like blood vessels appears inside the image of the avascular region. The third problem is thought to be caused by the fact that the multiscale Frangi filter enhances not only the images of blood vessels but also the noise, causing the noise in the avascular region to be depicted in a manner resembling blood vessels.

Each aspect example of the present disclosure aims to address at least one of these three problems. Some aspect examples may address a problem other than the three problems. Several non-limiting aspect examples of embodiment examples according to the present disclosure are listed below.

The first aspect example is an ophthalmic apparatus including an image acquisition unit, an image projection processor, a blood vessel enhancement processor, a denoising processor, and an image compositing processor. The image acquisition unit is configured to acquire an OCTA image of a fundus of a subject's eye. The image projection processor is configured to apply a projection process to the OCTA image, thereby generating a projection image. The blood vessel enhancement processor is configured to apply a blood vessel enhancing filter that is configured to enhance a blood vessel image to the projection image, thereby generating a blood vessel enhanced image. The denoising processor is configured to apply a denoising process to the projection image, thereby generating a denoised image. Here, the denoising process is a process for removing or reducing noise. The image compositing processor is configured to apply an image compositing process to the projection image, the blood vessel enhanced image, and the denoised image, thereby generating a composite image.

The second aspect example is the ophthalmic apparatus of the first aspect example, and the blood vessel enhancing filter includes a multiscale Frangi filter.

The blood vessel enhancing filter is not limited to a multiscale Frangi filter. The blood vessel enhancing filter may be an image filter of any type that can be used for processing focusing on blood vessel images, such as blood vessel detection, blood vessel enhancement, or other processing. The blood vessel enhancing filter may be a single image filter or a combination of two or more image filters.

The third aspect example is the ophthalmic apparatus of the first or second aspect example, and the projection process may include at least one of maximum intensity projection (hereinafter referred to as MIP) and average intensity projection (hereinafter referred to as AIP). MIP is well-suited for visualization of blood vessels, and is basically employed in the projection process of OCTA. On the other hand, a drawback of MIP is that it is prone to noise contamination. AIP is one of the projection methods that is less susceptible to noise contamination.

The projection process is not limited to MIP and AIP, and may be any type of projection process. The projection process may be a single projection process or a combination of two or more projection processes.

The fourth aspect example is the ophthalmic apparatus of any of the first to third aspect examples, in which the image projection processor is configured to apply a projection process to an image region in the OCTA image that corresponds to a predetermined a layer tissue of an eye fundus to generate a projection image.

The fundus layer tissue to which the projection process is applied may be, for example, the retina, one or more sub-tissues of the retina, the choroid, one or more sub-tissues of the choroid, or the sclera. The fundus layer tissue to which the projection process is applied is identified and extracted, for example, by the use of a segmentation process freely selected. The segmentation process used may be determined, selected, or configured depending on, for example, the type of the layer tissue to be identified, the type of projection process to be applied, the type or state of an OCTA image to which the segmentation process is applied, or other conditions.

The fifth aspect example is the ophthalmic apparatus of any of the first to fourth aspect examples, in which the denoising processor is configured to be able to perform a plurality of processes of mutually different types in the denoising process.

The denoising processor may be configured to select and perform one or more processes from the plurality of processes. Alternatively, the denoising processor may be configured to perform all of the plurality of processes.

The sixth aspect example is the ophthalmic apparatus of the fifth aspect example, in which the denoising processor is configured to select at least one process from the plurality of processes based on a field of view of the OCTA image. The denoising processor is further configured to generate the denoised image by applying the at least one selected process to the projection image. In addition, the image compositing processor is configured to generate the composite image by applying the image compositing process to the denoised image generated by applying the at least one selected process to the projection image, the projection image, and the blood vessel enhanced image.

The seventh aspect example is the ophthalmic apparatus of the fifth aspect example, in which the denoising processor is configured to select at least one process from the plurality of processes based on at least one of an eye fixation position and a scan area that are used for generating the OCTA image. Furthermore, the denoising processor is configured to generate the denoised image by applying the at least one selected process to the projection image. In addition, the image compositing processor is configured to generate the composite image by applying the image compositing process to the denoised image generated by applying the at least one selected process to the projection image, the projection image, and the blood vessel enhanced image.

The eighth aspect example is the ophthalmic apparatus of any of the first to seventh aspect examples, in which the image compositing process includes alpha blending. Alpha blending is a processing method for composing two or more images at a specific ratio.

The image compositing process is not limited to alpha blending, and may be any type of image compositing process. The image compositing process may be a single image compositing process or a combination of two or more image compositing processes.

The ninth aspect example is the ophthalmic apparatus of any of the first to eighth aspect examples, in which the image acquisition unit includes a scanner and an image constructing processor. The scanner is configured to collect data by applying OCT scanning to the fundus. The image constructing processor is configured to construct an OCTA image based on the data collected by the scanner.

The tenth aspect example is the ophthalmic apparatus of any of the first to ninth aspect examples, in which the image acquisition unit includes an image reception unit. The image reception unit receives an OCTA image from outside the ophthalmic apparatus. The OCTA image received by the image reception unit may be an image generated by the ophthalmic apparatus of the present aspect, or may be an image generated by another ophthalmic apparatus.

The eleventh aspect example is the ophthalmic apparatus of any of the first to tenth aspect examples, in which the image acquisition unit includes a data reception unit and an image constructing processor. The data reception unit is configured to receive data collected by applying OCT scanning to the fundus. The image constructing processor is configured to construct an OCTA image based on the data received by the data reception unit.

The data received by the data reception unit may be data generated by the ophthalmic apparatus of the present aspect, or may be data generated by another ophthalmic apparatus.

The first to eleventh aspect examples can be used to address various problems caused by image filters used for blood vessel enhancement, including the first problem (brightness irregularity in thick blood vessel images), the second problem (dropout of thin blood vessel images), and the third problem (noise in avascular region images), which are focused on by the present disclosure.

The twelfth aspect example is the ophthalmic apparatus of any of the first to eleventh aspect examples, in which the denoising processor is configured to apply, to the projection image, a blood vessel image extracting process that extracts a blood vessel image with a width belonging to a first range. The denoising processor is further configured to apply an erosion process to an image generated through the blood vessel image extracting process, thereby generating an eroded image in which a reduced blood vessel image corresponding to the extracted blood vessel image with a width reduced by the erosion process is depicted. Furthermore, the blood vessel enhancement processor is configured to apply a multiscale Frangi filter to the projection imagen thereby generating a first blood vessel enhanced image. The blood vessel enhancement processor is further configured to apply, to the projection image, a Frangi filter of a scale corresponding to a second range smaller than the first range and further apply gamma correction that increases brightness of a blood vessel image to an image generated by this Frangi filter, thereby generating a second blood vessel enhanced image. Moreover, the denoising processor is configured to generate the denoised image based on the eroded image, the first blood vessel enhanced image, and the second blood vessel enhanced image. In addition, the image compositing processor is configured to generate the composite image by applying the image compositing process to the projection image, the first blood vessel enhanced image, and the denoised image generated from the eroded image, the first blood vessel enhanced image, and the second blood vessel enhanced image.

The thirteenth aspect example is the ophthalmic apparatus of the twelfth aspect example, in which the denoising processor is configured to perform the following series of processes. First, the denoising processor identifies a first sub-image of the first blood vessel enhanced image that corresponds to the reduced blood vessel image in the eroded image. Second, the denoising processor identifies a second sub-image of the second blood vessel enhanced image that corresponds to the reduced blood vessel image in the eroded image. Third, the denoising processor generates the denoised image by selecting a higher brightness value between a brightness value of each pixel of the first sub-image and a brightness value of a corresponding pixel of the second sub-image. The image compositing processor is configured to generate the composite image by applying the image compositing process to the projection image, the first blood vessel enhanced image, and the denoised image generated from the first sub-image and the second sub-image.

The fourteenth aspect example is the ophthalmic apparatus of the thirteenth aspect example, in which the denoising processor is configured to determine the first sub-image by applying, to the first blood vessel enhanced image, a masking process based on the reduced blood vessel image in the eroded image. Furthermore, the denoising processor is configured to determine the second sub-image by applying the masking process to the second blood vessel enhanced image.

The fifteenth aspect example is the ophthalmic apparatus of any of the twelfth to fourteenth aspect examples, in which the projection process to generate the projection image to which the blood vessel image extracting process is applied includes MIP.

The sixteenth aspect example is the ophthalmic apparatus of any of the twelfth to fifteenth aspect examples, in which the blood vessel image extracting process includes Otsu's method (also referred to as Otsu's binarization, Otsu's thresholding, or the like).

The seventeenth aspect example is the ophthalmic apparatus of any of the first to sixteenth aspect examples, in which the denoising processor is configured to perform the following series of processes. First, the denoising processor applies, to the projection image, a blood vessel image extracting process that extracts a blood vessel image with a width belonging to a first range. Second, the denoising processor analyzes the blood vessel image extracted by the blood vessel image extracting process to determine a centerline of the blood vessel image. Third, the denoising processor determines a brightness profile (also referred to as brightness distribution) with respect to distance from the centerline. Fourth, the denoising processor applies a process based on the brightness profile to an image generated through the blood vessel image extracting process, thereby generating a processed image in which a reduced blood vessel image corresponding to the extracted blood vessel image with a reduced width is depicted. In addition, the blood vessel enhancement processor is configured to apply a multiscale Frangi filter to the projection image, thereby generating a first blood vessel enhanced image. Furthermore, the blood vessel enhancement processor is configured to apply, to the projection image, a Frangi filter of a scale corresponding to a second range smaller than the first range and further apply gamma correction that increases brightness of a blood vessel image to an image generated by this Frangi filter, thereby generating a second blood vessel enhanced image. The denoising processor is configured to generate the denoised image based on the processed image, the first blood vessel enhanced image, and the second blood vessel enhanced image. In addition, the image compositing processor is configured to generate the composite image by applying the image compositing process to the projection image, the first blood vessel enhanced image, and the denoised image generated from the processed image, the first blood vessel enhanced image, and the second blood vessel enhanced image.

The eighteenth aspect example is the ophthalmic apparatus of the seventeenth aspect example, in which the denoising processor is configured to perform the following series of processes. First, the denoising processor identifies a first sub-image of the first blood vessel enhanced image that corresponds to the reduced blood vessel image in the processed image. Second, the denoising processor identifies a second sub-image of the second blood vessel enhanced image that corresponds to the reduced blood vessel image in the processed image. Third, the denoising processor generates the denoised image by selecting a higher brightness value between a brightness value of each pixel of the first sub-image and a brightness value of a corresponding pixel of the second sub-image. The image compositing processor is configured to generate the composite image by applying the image compositing process to the projection image, the first blood vessel enhanced image, and the denoised image generated from the first sub-image and the second sub-image.

The nineteenth aspect example is the ophthalmic apparatus of the eighteenth aspect example, in which the denoising processor is configured to determine the first sub-image by applying, to the first blood vessel enhanced image, a masking process based on a reduced blood vessel image in the processed image. The denoising processor is further configured to determine the second sub-image by applying the masking process to the second blood vessel enhanced image.

The twentieth aspect example is the ophthalmic apparatus of any of the seventeenth to nineteenth aspect examples, in which the projection process to generate the projection image to which the blood vessel image extracting process is applied includes MIP.

The twenty-first aspect example is the ophthalmic apparatus of any of the seventeenth to twentieth aspect examples, in which the blood vessel image extracting process includes Otsu's method.

The twelfth to twenty-first aspect examples can be used primarily to address the first problem (irregularity in brightness in the images of thick blood vessels). Additionally, the twelfth to twenty-first aspect examples may also be used to address another problem caused by image filters used for blood vessel enhancement.

The twenty-second aspect example is the ophthalmic apparatus of any of the first to twenty-first aspect examples, in which the image projection processor is configured to apply a first projection process to the OCTA image, thereby generating a first projection image. The image projection processor is further configured to apply, to the OCTA image, a second projection process that is different from the first projection process, thereby generating a second projection image. Further, the blood vessel enhancement processor is configured to apply a multiscale Frangi filter to the first projection image to generate a first blood vessel enhanced image. The blood vessel enhancement processor is further configured to apply, to the second projection image, a Frangi filter of a scale corresponding to the range of a width of a capillary and further apply gamma correction that increases brightness of a blood vessel image to an image generated by this Frangi filter, thereby generating a second blood vessel enhanced image as the denoised image. In addition, the image compositing processor is configured to generate the composite image by applying the image compositing process to the first projection image, the first blood vessel enhanced image, and the second blood vessel enhanced image.

The twenty-third aspect example is the ophthalmic apparatus of the twenty-second aspect example, in which the first projection process is MIP. Furthermore, the second projection process is AIP.

The twenty-fourth aspect example is the ophthalmic apparatus of the twenty-second or twenty-third aspect example, in which the OCTA image is an image in which a region of the fundus including radial peripapillary capillaries (hereinafter referred to as RPCs) is depicted.

The twenty-second to twenty-fourth aspect examples can be used primarily to address the second problem (dropout of the image of thin blood vessels). Additionally, the twenty-second to twenty-fourth aspect examples may also be used to address another problem caused by image filters used for blood vessel enhancement.

The twenty-fifth aspect example is the ophthalmic apparatus of any of the first to twenty-fourth aspect examples, in which the blood vessel enhancement processor is configured to apply a multiscale Frangi filter to the projection image to generate a blood vessel enhanced image. The denoising processor is configured to apply, to the blood vessel enhanced image, avascular region identifying process that identifies an avascular region image corresponding to an avascular region of the fundus. The denoising processor is further configured to generate the denoised image by applying, to the blood vessel enhanced image, a masking process based on the avascular region image identified by the avascular region identifying process. Furthermore, the image compositing processor is configured to generate the composite image by applying the image compositing process to the projection image and the denoised image generated by applying the masking process based on the avascular region image to the blood vessel enhanced image.

The image compositing in the first aspect example (and the aspect examples referring thereto) is performed by combining three images, namely, the projection image, the blood vessel enhanced image, and the denoised image. On the other hand, the image compositing in the twenty-fifth aspect example is performed by combining two images, namely, the projection image, and the denoised image obtained from the blood vessel enhanced image. Here, since the denoised image in the twenty-fifth aspect example can be regarded as an image obtained by integrating the blood vessel enhanced image and the denoised image, the image compositing in the twenty-fifth aspect example corresponds to an embodiment or implementation of the image compositing in the first aspect example.

The twenty-sixth aspect example is the ophthalmic apparatus of the twenty-fifth aspect example, in which the denoising processor is configured to perform, in the avascular region identifying process, a first filtering process that applies a variance filter to the blood vessel enhanced image.

The twenty-seventh aspect example is the ophthalmic apparatus of the twenty-sixth aspect example, in which the denoising processor is configured to perform a first brightness threshold determining process and a first thresholding process in the avascular region identifying process. In the first brightness threshold determining process, the denoising processor determines a first brightness threshold based on a variance filtered image generated by the first filtering process. In the first thresholding process, the denoising processor applies a thresholding process with the first brightness threshold to the variance filtered image, thereby generating a first mask image.

The twenty-eighth aspect example is the ophthalmic apparatus of the twenty-sixth aspect example, in which the denoising processor is configured to perform, in the avascular region identifying process, a second filtering process that applies a mean filter to the projection image.

The twenty-ninth aspect example is the ophthalmic apparatus of the twenty-seventh aspect example, in which the denoising processor is configured to perform, in the avascular region identifying process, a second filtering process that applies a mean filter to the projection image.

The thirtieth aspect example is the ophthalmic apparatus of the twenty-ninth aspect example, in which the denoising processor is configured to perform a second brightness threshold determining process and a second thresholding process in the avascular region identifying process. In the second brightness threshold determining process, the denoising processor determines a second brightness threshold based on a mean filtered image generated by the second filtering process. In the second thresholding process, the denoising processor applies a thresholding process with the second brightness threshold to the mean filtered image, thereby generating a second mask image.

The thirty-first aspect example is the ophthalmic apparatus of the thirtieth aspect example, in which the denoising processor is configured to perform, in the avascular region identifying process, a process of composing the first mask image and the second mask image to generate a composite mask image, and a process of generating the avascular region image based on the composite mask image.

The thirty-second aspect example is the ophthalmic apparatus of the thirty-first aspect example, in which the denoising processor is configured to generate a summation image of the first mask image and the second mask image as the composite mask image in the avascular region identifying process.

The thirty-third aspect example is the ophthalmic apparatus of any of the twenty-fifth to thirty-second aspect examples, in which the denoising processor is configured to generate the avascular region image using a gaussian filter in the avascular region identifying process.

The thirty-fourth aspect example is the ophthalmic apparatus of the thirty-first aspect example, in which the denoising processor is configured to generate the avascular region image by applying a gaussian filter to the composite mask image in the avascular region identifying process.

The thirty-fifth aspect example is the ophthalmic apparatus of the thirty-second aspect example, in which the denoising processor is configured to generate the avascular region image by applying a gaussian filter to the summation image in the avascular region identifying process.

The thirty-sixth aspect example is the ophthalmic apparatus of any of the first to thirty-fifth aspect examples, in which the blood vessel enhancement processor is configured to apply a multiscale Frangi filter to the projection image, thereby generating a blood vessel enhanced image. Further, the denoising processor is configured to apply, to the projection image or the blood vessel enhanced image, a high density vascular region identifying process that identifies a high density vascular region image corresponding to a high density vascular region of the fundus. The denoising processor is further configured to generate the denoised image by applying, to the blood vessel enhanced image, a masking process based on the high density vascular region image identified by the high density vascular region identifying process. In addition, the image compositing processor is configured to generate the composite image by applying the image compositing process to the projection image and the denoised image generated by applying, to the blood vessel enhanced image, the masking process based on the high density vascular region image.

The thirty-seventh aspect example is the ophthalmic apparatus of the thirty-sixth aspect example, in which the denoising processor is configured to generate a mask image by expanding the high density vascular region image identified by the high density vascular region identifying process. Furthermore, the denoising processor is configured to generate the denoised image by applying a masking process using the mask image to the blood vessel enhanced image.

The twenty-fifth to thirty-seventh aspect examples can be used primarily to address the third problem (noise in an image of an avascular region). Additionally, the twenty-fifth to thirty-seventh aspect examples can also be used to address another problem caused by image filters used for blood vessel enhancement.

The thirty-eighth aspect example is a method of processing an ophthalmic image, more specifically, a method of processing an OCTA image of a fundus of a subject's eye by using a computer. The computer includes a processor, memory, and a data input interface. The method of the present aspect example includes an inputting process step, a storing process step, an image projection process step, a blood vessel enhancement process step, a denoising process step, and an image compositing process step. The inputting process step is performed by the data input interface to input the OCTA image into the computer. The storing process step is performed by the memory to store the OCTA image. The image projection process step is performed by the processor to apply a projection process to the OCTA image stored in the memory, thereby generating a projection image. The blood vessel enhancement process step is performed by the processor to apply, to the projection image, a blood vessel enhancing filter that is configured to enhance a blood vessel image, thereby generating a blood vessel enhanced image. The denoising process step is performed by the processor to apply a denoising process to the projection image, thereby generating a denoised image. The image compositing process step is performed by the processor to apply an image compositing process to the projection image, the blood vessel enhanced image, and the denoised image, thereby generating a composite image.

It may be possible to combine, with the method of the thirty-eighth aspect example, one or more steps that correspond, at least in part, to the features (such as configurations, operations, processes, or other aspects) according to any of the second to thirty-sixth aspect examples.

The thirty-ninth aspect example is a method of controlling an ophthalmic apparatus. The ophthalmic apparatus includes a processor, memory, and an image acquisition device. The method of the present aspect example includes an image acquisition control step, a storing control step, an image projection control step, a blood vessel enhancement control step, a denoising control step, and an image compositing control step. The image acquisition control step controls the image acquisition device to acquire an OCTA image of a fundus of a subject's eye. The storing control step controls the memory to store the OCTA image acquired. The image projection control step controls the processor to apply a projection process to the OCTA image stored in the memory, thereby generating a projection image. The blood vessel enhancement control step controls the processor to apply, to the projection image, a blood vessel enhancing filter that is configured to enhance a blood vessel image, thereby generating a blood vessel enhanced image. The denoising control step controls the processor to apply a denoising process to the projection image, thereby generating a denoised image. The image compositing control step controls the processor to apply an image compositing process to the projection image, the blood vessel enhanced image, and the denoised image, thereby generating a composite image.

It may be possible to combine, with the method of the thirty-ninth aspect example, one or more steps that correspond, at least in part, to the features (such as configurations, operations, processes, or other aspects) according to any of the second to thirty-sixth aspect examples.

The fortieth aspect example is a program configured to cause a computer to execute each step in the method of the thirty-eighth or thirty-ninth aspect example.

It may be possible to combine, with the program of the fortieth aspect example, one or more program elements configured to cause the computer to execute one or more steps that correspond, at least in part, to the features (such as configurations, operations, processes, or other aspects) according to any of the second to thirty-sixth aspect examples.

The forty-first aspect example is a computer-readable non-transitory recording medium storing the program of the fortieth aspect example.

The recording medium of the forty-first aspect example may be configured to store a program including one or more program elements configured to cause the computer to execute one or more steps that correspond, at least in part, to the features (such as configurations, operations, processes, or other aspects) according to any of the second to thirty-sixth aspect examples.

The thirty-eighth to forty-first aspect examples can be used to address various problems caused by image filters used for blood vessel enhancement, including the first problem (irregularity in brightness in the images of thick blood vessels), the second problem (dropout of the image of thin blood vessels), and the third problem (noise in the image of the avascular region). It is also possible to select one or more elements to be combined with each aspect example depending on a problem of particular interest.

The present disclosure describes various non-limiting aspects, including the first to forty-first aspects mentioned above. The present disclosure mainly describes some non-limiting aspects of an ophthalmic apparatus (such as an ophthalmic imaging apparatus, an ophthalmic image processing apparatus, or other aspects), some non-limiting aspects of a method of processing an ophthalmic image, some non-limiting aspects of a method of controlling an ophthalmic apparatus, some non-limiting aspects of a program, and some non-limiting aspects of a recording medium. The categories of aspects of embodiment examples are not limited to these non-limiting categories of aspects. For example, it would be understandable to a person skilled in the art in each technical field that the embodiment examples according to the present disclosure can provide various aspects in any other categories such as: devices, systems, methods, programs, recording media, etc. related to medical fields other than ophthalmology; and devices, systems, methods, programs, recording media, etc. related to technical fields other than medical field.

### Embodiment Example of Ophthalmic Apparatus

Several non-limiting aspects of the ophthalmic apparatus according to embodiment examples will be described. The ophthalmic apparatus according to the embodiment examples has the function of generating an OCTA image or the function of acquiring an OCTA image from an external source, and the function of processing the generated or acquired OCTA image.

An ophthalmic apparatus of the aspects mainly described in the present disclosure is configured to function as an OCT apparatus capable of performing OCTA (including OCT scanning and image constructing process). An ophthalmic apparatus of other aspects may not be capable of performing at least one of the OCT scanning and image constructing process.

The OCT method may be freely selected, and may be, for example, either spectral domain OCT or swept source OCT. Spectral domain OCT is a technique including the following processes: a process of splitting light emitted by a low coherence light source into measurement light and reference light; a process of generating interference light by superposing return light of the measurement light from a sample and the reference light; a process of detecting a spectral distribution (spectral components) of the interference light by a spectrometer; and a process of constructing an image of the sample by applying signal processing including a Fourier transform to the spectral distribution detected. Swept source OCT is a technique including the following processes: a process of splitting light emitted by a wavelength tunable light source into measurement light and reference light; a process of generating interference light by superposing return light of the measurement light from a sample and the reference light; a process of detecting the interference light by a photodetector (such as a balanced photodiode); and a process of constructing an image of the sample by applying signal processing including a Fourier transform to detection data collected corresponding to wavelength sweeping (change in emitted wavelengths) and scanning with the measurement light. In short, spectral domain OCT can be said to be an OCT method of acquiring a spectral distribution in a space-divisional manner while swept source OCT can be said to be an OCT method of acquiring a spectral distribution in a time-divisional manner. Note that other OCT methods such as time domain OCT may also be employed.

The ophthalmic apparatus of the aspect mainly described in the present disclosure has the function as a fundus camera capable of imaging the fundus, or may have the function as any type of ophthalmic imaging modality, such as a scanning laser ophthalmoscope (SLO), a slit lamp microscope, or a surgical microscope.

The term “image data” and the term “image”, which is visual information formed based on this image data, are not distinguished from each other in the present disclosure unless otherwise mentioned. Also, a site or tissue of a subject's eye and an image (or image data) of this site or tissue are not distinguished from each other in the present disclosure unless otherwise mentioned.

### Configuration of Ophthalmic Apparatus

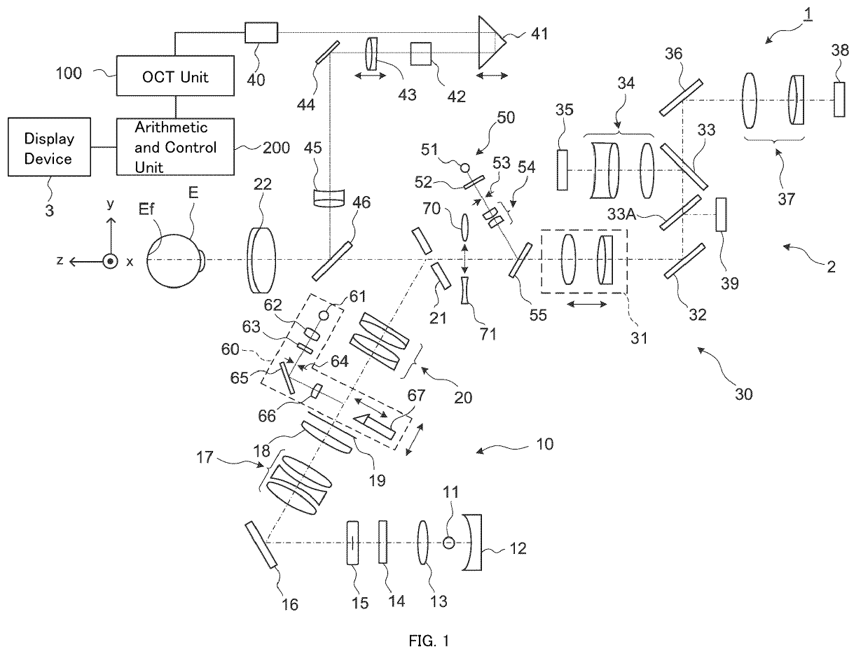

The configuration of the ophthalmic apparatus according to a non-limiting embodiment example is shown in FIGS. 1 to 3. The ophthalmic apparatus 1 includes the fundus camera unit 2, the OCT unit 100, and the arithmetic and control unit 200. The fundus camera unit 2 is provided with elements of a fundus camera that is capable of eye fundus imaging and anterior segment imaging, as well as some elements of an OCT scanner. The OCT unit 100 includes some other elements of the OCT scanner. The arithmetic and control unit 200 includes one or more processors that are configured to execute various processes such as calculation, analysis, control, and other types of processes.

One or more of the functions of the elements of the embodiment examples according to the present disclosure can be implemented by using a circuit configuration (circuitry) or a processing circuit configuration (processing circuitry). The circuitry or the processing circuitry includes any of the followings, all of which are configured and/or programmed to execute one or more functions disclosed herein: a general purpose processor, a dedicated processor, an integrated circuit, a central processing unit (CPU), a graphics processing unit (GPU), an application specific integrated circuit (ASIC), a programmable logic device (e.g., a simple programmable logic device (SPLD), a complex programmable logic device (CPLD), or a field programmable gate array (FPGA)), a conventional circuit configuration or circuitry, and any combination of these. A processor is considered to be processing circuitry or circuitry that includes a transistor and/or another circuitry. In the present disclosure, circuitry, a unit, a means, or any terms similar to these is hardware configured to execute one or more functions disclosed herein, or hardware that is programmed to execute one or more functions disclosed herein. The hardware may be any hardware disclosed in the present specification, or alternatively, known hardware that is programmed and/or configured to execute one or more functions described herein. In the case where the hardware is a processor, which may be considered as a certain type of circuitry, then circuitry, a unit, a means, or any terms similar to these is a combination of hardware and software. In this case, the software is used to configure the hardware and/or the processor.

### Fundus Camera Unit 2

The fundus camera unit 2 includes an optical system for photographing the fundus Ef (and the anterior segment) of the subject's eye E. Digital images acquired by the fundus camera unit 2 are typically front images (en face images). The fundus camera unit 2 can acquire an observation image by performing video recording using, for example, near-infrared constant light as illumination light. The fundus camera unit 2 can acquire a photographed image by performing photographing using visible flash light as illumination light.

The fundus camera unit 2 includes the illumination optical system 10 and the photographing optical system 30. The illumination optical system 10 projects illumination light onto the subject's eye E. The photographing optical system 30 detects return light of the illumination light projected onto the subject's eye E. Measurement light provided from the OCT unit 100 is directed to the subject's eye E through an optical path in the fundus camera unit 2. Return light of this measurement light projected onto the subject's eye E is directed to the OCT unit 100 through an optical path in the fundus camera unit 2.

Observation illumination light emitted by the observation light source 11 of the illumination optical system 10 is reflected by the concave mirror 12, passes through the condenser lens 13, and becomes near-infrared light after passing through the visible cut filter 14. Further, the observation illumination light is once converged at a location near the photographing light source 15, reflected by the mirror 16, and passes through the relay lens system 17, the relay lens 18, the diaphragm 19, and the relay lens system 20. Then, the observation illumination light is directed to the aperture mirror 21, reflected on the mirror part surrounding the central aperture part of the aperture mirror 21, penetrates the dichroic mirror 46, is refracted by the objective lens 22, and is projected onto the subject's eye E (and onto the fundus Ef). Return light of the observation illumination light projected onto the subject's eye E is refracted by the objective lens 22, penetrates the dichroic mirror 46, passes through the central aperture part of the aperture mirror 21, passes through the dichroic mirror 55, travels through the photography focusing lens 31, and is reflected by the mirror 32. Furthermore, the return light of the observation illumination light passes through the half mirror 33A, is reflected by the dichroic mirror 33, and forms an image on the light receiving surface of the image sensor 35 by the imaging lens 34. The image sensor 35 detects the return light at a predetermined time interval (time rate). It should be noted that the focus of the photographing optical system 30 is adjusted depending on the site to be photographed.

Photographing illumination light emitted by the photographing light source 15 passes through the same route as the route of the observation illumination light and is projected onto the fundus Ef. Return light of the photographing illumination light from the subject's eye E passes through the same route as the route of the return light of the observation illumination light to the dichroic mirror 33, passes through the dichroic mirror 33, is reflected by the mirror 36, and forms an image on the light receiving surface of the image sensor 38 by the imaging lens 37.

The liquid crystal display (hereinafter referred to as LCD) 39 displays a fixation target (fixation target image) for guiding and fixing the line of sight. A light beam output from the LCD 39 is reflected by the half mirror 33A and the mirror 32, travels through the photography focusing lens 31 and the dichroic mirror 55, and passes through the central aperture part of the aperture mirror 21. The light beam having passed through the central aperture part of the aperture mirror 21 penetrates the dichroic mirror 46, and is refracted by the objective lens 22, thereby being projected onto the fundus Ef. This allows the subject to visually recognize the fixation target.

The alignment optical system 50 generates an alignment indicator used for alignment of the ophthalmic apparatus 1 with respect to the subject's eye E. Alignment light emitted by the light emitting diode (hereinafter referred to as LED) 51 travels through the diaphragm 52, the diaphragm 53, and the relay lens 54, is reflected by the dichroic mirror 55, passes through the central aperture part of the aperture mirror 21, penetrates the dichroic mirror 46, and is projected onto the subject's eye E via the objective lens 22. Return light of the alignment light from the subject's eye E passes through the same route as the route of the return light of the observation illumination light and is guided to the image sensor 35. Manual alignment and/or automatic alignment can be performed by referring to an image detected by the image sensor 35 (alignment indicator image).

The focusing optical system 60 generates a split indicator used for focus adjustment (focusing, focusing operation) with respect to the subject's eye E. The focusing optical system 60 is moved along the optical path of the illumination optical system 10 in conjunction with movement of the photography focusing lens 31 along the optical path of the photographing optical system 30. The optical path of the illumination optical system 10 is referred to as the illumination optical path, and the optical path of the photographing optical system 30 is referred to as the photographing optical path. The reflection rod 67 is inserted into and removed from the illumination optical path. The reflective surface of the reflection rod 67 is inserted into the illumination optical path and placed in an oblique orientation before performing focus adjustment. Focus light emitted by the LED 61 passes through the relay lens 62, is split into two light beams by the split indicator plate 63, and passes through the two-hole diaphragm 64. The focus light, then, is reflected by the mirror 65, is converged on the reflective surface of the reflection rod 67 by the condenser lens 66, and is reflected by the reflective surface. Further, the focus light travels through the relay lens 20, is reflected by the aperture mirror 21, and penetrates the dichroic mirror 46, thereby being projected onto the subject's eye E via the objective lens 22. Return light of the focus light from the subject's eye E passes through the same route as the route of the return light of the alignment light and is guided to the image sensor 35. Manual focusing and/or automatic focusing can be performed by referring to an image detected by the image sensor 35 (split indicator image).

The diopter correction lenses 70 and 71 are selectively inserted into the photographing optical path between the aperture mirror 21 and the dichroic mirror 55. The diopter correction lens 70 (positive lens) is used for correcting high hyperopia. The diopter correction lens 71 (negative lens) is used for correcting high myopia.

The dichroic mirror 46 combines the optical path for fundus imaging and the optical path for OCT scanning. The optical path for OCT scanning is referred to as a sample arm. The dichroic mirror 46 reflects light of wavelength bands used for OCT scanning while transmitting light for fundus imaging. Listed from the OCT unit 100 side, the sample arm includes the collimator lens unit 40, the retroreflector 41, the dispersion compensation member 42, the OCT focusing lens 43, the optical scanner 44, and the relay lens 45. The retroreflector 41 is movable along the optical path of the measurement light LS incident onto the retroflector 41, and may be used for operations such as optical path length correction on the basis of eye axial length and adjustment or regulation of interference conditions or states. The dispersion compensation member 42 is used for dispersion compensation between the sample arm and the reference arm. The OCT focusing lens 43 is movable along the sample arm, and may be used for focus adjustment of the sample arm. The focus adjustment of the ophthalmic apparatus 1 is performed by a combined operation between the movement of the photography focusing lens 31, the movement of the focusing optical system 60, and the movement of the OCT focusing lens 43. The optical scanner 44 is placed at a position substantially conjugate with the pupil of the subject's eye E by alignment, and functions to change the traveling direction of the measurement light LS. In some examples, the optical scanner 44 may be a galvanometer scanner configured to be capable of two-dimensional scanning.

### OCT Unit 100

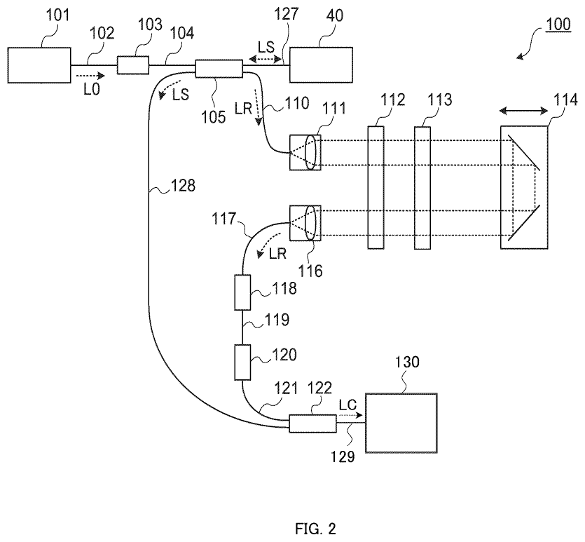

As illustrated in FIG. 2, the OCT unit 100 is provided with a spectral-domain type OCT optical system. This OCT optical system includes an interference optical system. This interference optical system is configured to split light emitted by a low coherence light source (broad band light source, wide band light source) into the measurement light LS and the reference light LR, and to superpose return light of the measurement light LS projected onto the subject's eye E and the reference light LR that has been guided by a reference optical path (reference arm), thereby generating interference light LC. The interference light LC thus generated is detected by the spectrometer 130. This provides a signal indicating the spectral distribution of the interference light LC. This detection signal is sent to the arithmetic and control unit 200.

The light source unit 101 outputs broadband low coherence light L0. The light source unit 101 includes a freely selected type of light-emitting device such as a super luminescent diode (SLD), an LED, or a semiconductor optical amplifier (SOA).

The low coherence light L0 output from the light source unit 101 is guided to the polarization controller 103 through the optical fiber 102. The polarization controller 103 is configured to perform regulation (adjustment) of the polarization condition (polarization state) of the light L0. Further, the light L0 is guided to the fiber coupler 105 through the optical fiber 104. The fiber coupler 105 is configured to split the light L0 into the measurement light LS and the reference light LR. The measurement light LS is guided by the sample arm, and the reference light LR is guided by the reference arm.

The reference light LR is guided through the optical fiber 110 to the collimator 111, is converted into a parallel light beam by the collimator 111, and travels through the optical path length correction member 112 for compensating for the optical distance between the sample arm and the reference arm. The reference light LR, then, travels through the dispersion compensation member 113 for dispersion compensation between the sample arm and the reference arm, and is guided to the retroreflector 114. The retroreflector 114 is movable along the optical path of the reference light LR that is incident onto the retroreflector 114, and may be used for operations such as optical path length correction on the basis of eye axial length and adjustment or regulation of interference conditions or states. The reference light LR that has passed through the retroreflector 114 travels through the dispersion compensation member 113 and the optical path length correction member 112, is converted from a parallel light beam to a convergent light beam by the collimator 116, is guided through the optical fiber 117 to the polarization controller 118, and the polarization state of the reference light LR is regulated by the polarization controller 118. The reference light LR output from the polarization controller 118 is guided to the attenuator 120 through the optical fiber 119, and the amount of light of the reference light LR is regulated by the attenuator 120. Subsequently, the reference light LR is guided to the fiber coupler 122 through the optical fiber 121.

Meanwhile, the measurement light LS generated by the fiber coupler 105 is guided through the optical fiber 127 to the collimator lens unit 40 and is converted into a parallel light beam by the collimator lens unit 40. The measurement light LS output from the collimator lens unit 40 passes through the retroreflector 41, the dispersion compensation member 42, the OCT focusing lens 43, the optical scanner 44, and the relay lens 45, is reflected by the dichroic mirror 46, and is refracted by the objective lens 22, thereby being projected onto the subject's eye E. The measurement light LS is reflected and scattered at various depth positions of the subject's eye E. Return light of the measurement light LS from the subject's eye E travels along the sample arm in the opposite direction to the fiber coupler 105, and then reaches the fiber coupler 122 through the optical fiber 128.

The fiber coupler 122 superposes the measurement light LS reached here through the optical fiber 128 and the reference light LR reached here through the optical fiber 121, thereby generating interference light LC. The interference light LC generated by the fiber coupler 122 is guided to the spectrometer 130 through the optical fiber 129. The spectrometer 130 of some examples converts the incident interference light LC into a parallel light beam by using a collimator lens, resolves the interference light LC converted into the parallel light beam into a plurality of spectral components by using a diffraction grating, and projects the plurality of spectral components generated by the diffraction grating onto an image sensor by using the lens 114. This image sensor is, for example, a line sensor that detects the plurality of spectral components of the interference light LC to generate an electrical signal (detection signal). The detection signal generated includes information on the spectral distribution of the interference light LC and is sent to the arithmetic and control unit 200.

In the case where the swept source OCT method is used instead of the spectral domain OCT method, the light source unit 101 of some examples includes a wavelength tunable light source (e.g., a near-infrared wavelength tunable laser) that changes the wavelength of the emitted light at high speed. In addition, the optical system of the swept source OCT method is configured to split, at a predetermined splitting ratio (e.g., 1 to 1), the interference light LC generated by superposing the measurement light LS and the reference light LR to generate a pair of interference light, and detect the pair of interference light by a photodetector. The photodetector includes a balanced photodiode. This balanced photodiode includes a pair of photodetectors that detects the pair of the interference light respectively. The balanced photodiode outputs a difference signal between a pair of detection signals corresponding to the pair of the interference light LC respectively obtained by the pair of photodetectors. The photodetector sends this difference signal to a data acquisition system (DAQ). A sampling clock is supplied to the data acquisition system from the light source unit 101. The clock is generated in the light source unit 101 in synchronization with the output timings of individual wavelengths varied over a predetermined wavelength range by the wavelength tunable light source. The light source unit 101 of some examples is configured to split the light of the individual output wavelengths to generate two pieces of split light, to apply an optical delay to one of the two pieces of split light, to superpose the resulting two pieces of split light with one another, to detect the resulting superposed light, and to generate the clock based on the detection signal of the superposed light. Based on the clock, the data acquisition system performs sampling of the detection signal (difference signal) input from the photodetector. The data obtained by this sampling is used for processing such as image construction.

In the examples shown in FIGS. 1 and 2, both the sample arm and the reference arm are provided with the optical path length changing element, namely, the retroreflector 41 for the sample arm and the retroreflector 114 for the reference arm. In some other aspect examples, only either one of these two elements may be provided. The optical path length changing element is not limited to a retroreflector. In some examples, the optical path length changing element of the reference arm may be a movable reflecting member (such as a reference mirror). More generally, some aspect examples include an element that is configured to cause relative changes between the sample arm length and the reference arm length, in other words, an element that is configured to cause changes in the optical path length difference between the sample arm and the reference arm, thereby allowing the coherence gate position to be moved.

### Arithmetic and Control Unit 200

The arithmetic and control unit 200 is configured to perform control of each part of the ophthalmic apparatus 1, various calculation processes, and various analysis processes. In some examples, the arithmetic and control unit 200 may be configured to apply signal processing including a Fourier transform to the spectral distribution acquired by the spectrometer 130, thereby calculating a reflection intensity profile along a line (A-line) extending in the depth direction (z-direction) at each projection position of the signal light LS. Furthermore, the arithmetic and control unit 200 may be configured to generate image data by executing an image construction process on the reflection intensity profile of each A-line. The arithmetic processing for this purpose may be performed in the same manner as the image construction technique of conventional spectral domain OCT methods. The arithmetic and control unit 200 of some examples includes a processor, RAM, ROM, a hard disk drive, and a communication interface. Various computer programs are stored in the memory such as the hard disk drive. The arithmetic and control unit 200 may further include an operation device, an input device, and a display device.

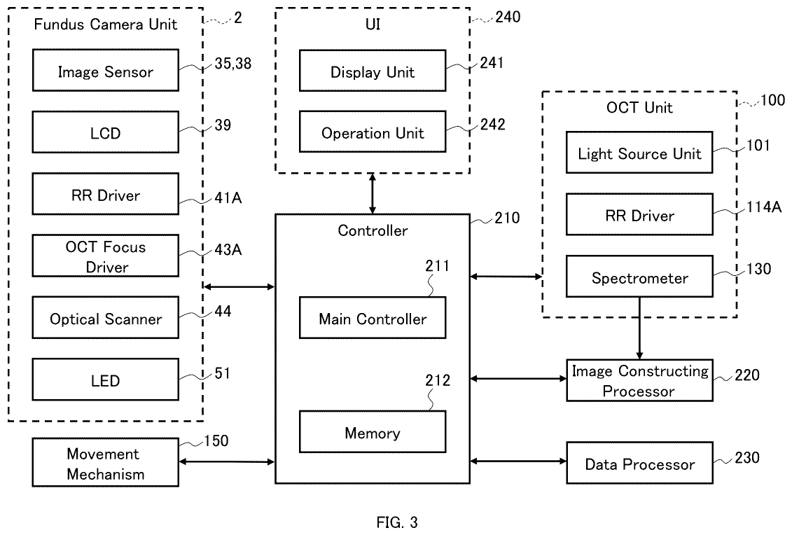

As shown in FIG. 3, the user interface 240 includes the display unit 241 and the operation unit 242. The display unit 241 may include the display device 3 illustrated in FIG. 1. The operation unit 242 may include freely selected types of operation devices and freely selected types of input devices. The user interface 240 may include a touch panel. In some aspect examples, at least part of the user interface is provided as a peripheral device that is connected to the ophthalmic apparatus 1. The movement mechanism 150 is configured to move the optical system of the ophthalmic apparatus 1, and some examples thereof is configured to move at least the fundus camera unit 2 in a three-dimensional manner.

### Processing System

The ophthalmic apparatus 1 includes the controller 210, the image constructing processor 220, and the data processor 230. These elements are provided in the arithmetic and control unit 200.

### Controller 210

The controller 210 includes a processor and configured to execute control of each part of the ophthalmic apparatus 1. The controller 210 includes the main controller 211 and the memory 212.

### Main Controller 211

The main controller 211 includes a processor and is configured to execute control of each element of the ophthalmic apparatus 1 (including control of each element shown in FIGS. 1 to 3). The main controller 211 may also be configured to be able to control an apparatus, device, or system connected to the ophthalmic apparatus 1. In some examples, the function of the main controller 211 is implemented by cooperation between hardware including a circuit and control software.

### Memory 212

The memory 212 stores various types of data. The memory 212 includes computer data storage (digital data storage) such as a hard disk drive, a solid-state drive, or other forms of storage.

### Image Constructing Processor 220

The image constructing processor 220 is configured to process data collected by applying OCT scanning to the fundus Ef of the subject's eye E, to generate OCT image data. The image constructing processor 220 includes a processor. In some examples, the function of the image constructing processor 220 is implemented by cooperation between hardware including a circuit and image construction software.

The image constructing processor 220 can construct cross sectional image data based on data acquired by the spectrometer 130. This image construction process includes signal processing such as sampling (A/D conversion), denoising (noise removal, noise reduction), filtering, fast Fourier transform (FFT), and other processes, as in existing or conventional spectral domain OCT methods.

The OCT image data constructed by the image constructing processor 220 is a data set that includes a group of image data (a group of A-scan image data). The group of image data is generated by executing image construction (visualization) on a plurality of reflection intensity profiles respectively corresponding to a plurality of A-lines that is arranged in the area to which the OCT scanning has been applied.

The OCT image data in some examples may be stack data constructed by embedding a plurality of B-scan image data in a single three-dimensional coordinate system. The image constructing processor 220 may construct volume data, which is also referred to as voxel data, by applying voxelization processing to the stack data. The stack data and the volume data are typical examples of three-dimensional image data that is image data represented using a three-dimensional coordinate system.

The image constructing processor 220 may be configured to process three-dimensional image data. The image constructing processor 220 of some examples may be configured to apply a rendering process to the three-dimensional image data to construct another form of image data. Possible techniques of the rendering include volume rendering, surface rendering, multi planar reconstruction (MPR), MIP, minimum intensity projection (MinIP), and AIP. The image constructing processor 220 may be configured to construct projection data of three-dimensional image data by means of projection (integration, addition) of the three-dimensional image data in the z-direction. The image constructing processor 220 may be configured to construct a shadowgram by projecting partial data of the three-dimensional image data (referred to as three-dimensional sub-image data) in the z-direction. The three-dimensional sub-image data is extracted from the three-dimensional image data by using a freely selected or determined segmentation method.

The ophthalmic apparatus 1 may be configured to be capable of performing OCTA. In OCTA, the ophthalmic apparatus 1 performs repetitive scanning targeting the same region of the fundus Ef a predetermined number of times. The image constructing processor 220 is configured to construct a motion contrast image based on difference information that is contained in the data set collected by the repetitive scanning. This motion contrast image is an image generated by enhancing and visualizing the interference signal that changes over time due to blood flow in fundus vessels, and is an angiographic image that represents the distribution of blood vessels in the fundus Ef (in fact, the distribution of blood flow). Typically, the ophthalmic apparatus 1 is configured to apply OCTA to a three-dimensional region of the fundus Ef to generate a plurality of pieces of three-dimensional data (i.e., data set), and generate three-dimensional angiographic image data that represents the three-dimensional distribution of fundus vessels based on this data set.

The image constructing processor 220 is configured to be capable of constructing any form of two-dimensional angiographic image data and/or any form of pseudo three-dimensional angiographic image data from the three-dimensional angiographic image data. In some examples, the image constructing processor 220 is capable of applying multi planar reconstruction to the three-dimensional angiographic image data, thereby constructing two-dimensional angiographic image data representing a freely selected or determined cross section of the fundus Ef. The image constructing processor 200 may be configured to construct front image data (en face image data) from an image region (slab) corresponding to a predetermined tissue that has been identified by applying segmentation to the three-dimensional angiographic image data. This front image data is an example of a shadowgram. Typically, multiple pieces of front image data are constructed for various depth areas such as the superficial retinal layer, the deep retinal layer, and the choroid. OCTA techniques are described in, for example, Japanese Unexamined Patent Application Publication No. 2019-42264 filed by the present applicant. The ophthalmic apparatus 1 may be configured to execute, by using the data processor 230, at least part of the processes related to OCTA.

### Data Processor 230

The data processor 230 may be configured to perform various kinds of data processing. In some examples, the data processor 230 may be configured to apply various processes to images acquired by the fundus camera unit 2 (such as fundus images and anterior segment images) and images generated by using OCT scanning (such as OCT images). The data processor 230 includes a processor. The data processor 230 of some examples is implemented by cooperation between hardware including a circuit and data processing software.

### Functional Configuration of Ophthalmic Apparatus

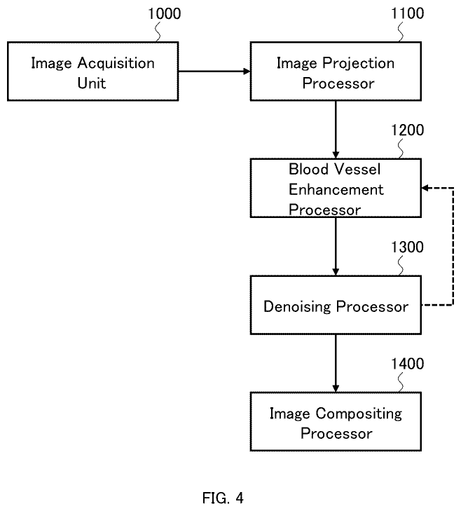

The functional configuration of the ophthalmic apparatus 1 implemented by the elements (including hardware elements and software elements) shown in FIGS. 1 to 3 will be described. FIG. 4 shows an example of the functional configuration of the ophthalmic apparatus 1.

The ophthalmic apparatus 1 according to FIG. 4 includes the image acquisition unit 1000, the image projection processor 1100, the blood vessel enhancement processor 1200, the denoising processor 1300, and the image compositing processor 1400.

### Image Acquisition Unit 1000

The image acquisition unit 1000 is configured to acquire an OCTA image of the fundus Ef of the subject's eye E. Some non-limiting examples of the image acquisition unit 1000 are described below.

An OCTA image acquired by the image acquisition unit 1000 may be in any form. In some examples, the OCTA image may be a three-dimensional OCTA image that represents a three-dimensional region of the fundus Ef. The three-dimensional OCTA image in some examples may be any of the following forms of image data: a data set consisting of a plurality of A-scan angiographic images; stack data constructed based on a plurality of A-scan angiographic images; a data set consisting of a plurality of B-scan angiographic images; stack data constructed based on a plurality of B-scan angiographic images; volume data constructed from stack data based on a plurality of A-scan angiographic images; and volume data constructed from stack data based on a plurality of B-scan angiographic images. Alternatively, three-dimensional OCTA image may be image data of a form other than the above forms.

According to the configuration shown in FIGS. 1 to 3, the image acquisition unit 1000 can be implemented by combining the fundus camera unit 2 (particularly, a group of elements forming a sample arm), the OCT unit 100, the image constructing processor 220, and the main controller 211 that controls these components. In this case, the image acquisition unit 1000 includes a scanner (including the fundus camera unit 2, the OCT unit 100, and the main controller 211) that is configured to apply OCT scanning to the fundus Ef to collect data, and the image constructing processor 220 that is configured to construct an OCTA image based on the data collected by the scanner.

In another example, the image acquisition unit 1000 may be implemented by the communication interface (described above) included in the arithmetic and control unit 200. In the image acquisition unit 1000 of the present example, the communication interface functions as an image reception unit that receives an OCTA image from outside the ophthalmic apparatus 1. The image acquisition unit 1000 of the present example may be configured to acquire (receive) an OCTA image stored in an external device via a communication line. Examples of the external device include an image archiving system, an ophthalmic imaging apparatus, memory, and systems or devices of other forms. The image acquisition unit 1000 of a similar example may include a drive device that reads out an OCTA image recorded on a recording medium. In the example shown in FIG. 1, the drive device may be provided in the arithmetic and control unit 200.

In yet another example, the image acquisition unit 1000 may include a data reception unit that receives data collected by applying OCT scanning to the fundus Ef, and the image constructing processor 220 that constructs an OCTA image based on the data received by the data reception unit. Examples of the data reception unit include the communication interface or the drive device mentioned above.

### Image Projection Processor 1100

The image projection processor 1100 is configured to apply a projection process to the OCTA image acquired by the image acquisition unit 1000 to generate a projection image.

For example, the image projection processor 1100 may be configured to project a three-dimensional OCTA image representing a three-dimensional region of the fundus Ef in a specific direction (e.g., the z-direction), thereby generating a two-dimensional OCTA image defined in the plane perpendicular to the projection direction (e.g., the xy-plane).

The projection direction may be determined in advance. Alternatively, the projection direction may be determined based on a freely selected condition, such as any of the following: the type, feature, characteristic (property), or other information of the three-dimensional OCTA image to which the projection process is applied; the site of the fundus Ef represented by the three-dimensional OCTA image; the type, parameter, or other information of the projection process; the type, feature, characteristic (property), or other information of a resulting image (e.g., the two-dimensional OCTA image) to be generated by using the projection process.

The present disclosure describes in detail some exemplary cases where MIP and/or AIP are/is employed as the projection process. The types of the projection process that can be adopted in embodiment examples are not limited to those used in the exemplary cases.

The projection process may be applied to the entirety of the OCTA image acquired by the image acquisition unit 1000, or to only a part of the OCTA image. In the latter case, for example, the image projection processor 1100 applies segmentation to the OCTA image acquired by the image acquisition unit 1000 to extract an image region corresponding to a specific layer tissue in the fundus Ef, and then applies the projection process to the extracted image region to generate a projection image. This layer tissue is typically at least a part of the retina; however, the layer tissue may be at least a part of the choroid, at least a part of the sclera, at least a part of the vitreous body, the gap between the retina and the vitreous body, or a combination of two or more of these.

According to the configuration shown in FIGS. 1 to 3, the image projection processor 1100 may be implemented by the use of any one or both of the image constructing processor 220 and the data processor 230.

### Blood Vessel Enhancement Processor 1200

The blood vessel enhancement processor 1200 is configured to apply a blood vessel enhancing filter configured to enhancing the blood vessel image to the projection image generated by the image projection processor 1100, thereby generating a blood vessel enhanced image. The blood vessel enhanced image is an image in which the blood vessel image in the projection image is represented in an enhanced manner.

The present disclosure provides a detailed description of some exemplary cases in which a multiscale Frangi filter is used as a blood vessel enhancing filter. A person skilled in the art would understand that the methods and techniques according to the present disclosure are also effective in the cases where image filters of other types (e.g., a Gabor filter, a non-local means filter, a wavelet filter, etc.) are employed.

It should be noted that non-limiting examples of image processing methods that may be used for the blood vessel enhancement of the embodiment examples include the following: a method using vector concentration based on concentration gradients; a method using a black top-hat transformation; a method using a double ring filter; a method combining a black top-hat transformation and a double ring filter; a method for detecting ridge lines of intensity values from the green channel of a color image; a method using Gabor wavelets; a method using matched filters; a method using a Hough transform; a method using a Contourlet transform; a method using a Curvelet transform; a method based on ensemble learning; a method using a morphological filter bank; and a method using machine learning.

### Denoising Processor 1300

The denoising processor 1300 is configured to apply a denoising process to the projection image generated by the image projection processor 1100 to generate a denoised image.

The denoising process executed by the denoising processor 1300 refers not only to a process for removing noise, but also to a process for reducing noise.

The denoising processor 1300 may be configured to be able to execute a plurality of processes of mutually different types in the denoising process. The plurality of processes in the denoising process may include the processes according to the three embodiment examples described below, namely, the first, second, and third embodiment examples mainly focusing on addressing the first, second, and third problems, respectively, described above.

In the case where the denoising processor 1300 is capable of performing the plurality of processes, the denoising processor 1300 may be configured to select at least one process from the plurality of processes based on the field of view of the OCTA image acquired by the image acquisition unit 1000. In other words, the denoising processor 1300 may be configured to select at least one process from the plurality of processes based on the size (dimension) of the area (scan area) of the OCT scanning that has been applied to the fundus Ef to generate the OCTA image. The denoising processor 1300 may be configured to be able to generate the denoised image by applying each process selected from the plurality of processes to the projection image generated by the image projection processor 1100.

For example, in the case of processing an OCTA image with a wide field of view that includes both the optic nerve head and the macula of the fundus Ef, this OCTA image can include images of thick blood vessels around the optic nerve head, images of thin blood vessels (capillaries) in various sites, and an image of the FAZ. In this case, the denoising processor 1300 may perform the following processes: the processes according to the below-mentioned first embodiment example that addresses the problem of irregularity in brightness in the images of thick blood vessels (the first problem); the processes according to the below-mentioned second embodiment example that addresses the problem of dropout of thin blood vessels (the second problem); and the processes according to the below-mentioned third embodiment example that addresses the problem of noise in the avascular region (the third problem).

In some other examples of processing an OCTA image with a narrow field of view that includes only the optic nerve head and its surroundings, thick blood vessels around the optic nerve head are depicted in the OCTA image. In this case, the denoising processor 1300 may perform the processes according to the below-mentioned first embodiment example that addresses the problem of irregularity in brightness in the images of thick blood vessels (the first problem). In addition, the denoising processor 1300 may also perform the processes according to the below-mentioned second embodiment example that addresses the problem of dropout of thin blood vessels (the second problem).

Yet in some other examples of processing an OCTA image with a narrow field of view that includes only the macula and its surroundings, the FAZ is depicted in the OCTA image. In this case, the denoising processor 1300 may perform the processes according to the below-mentioned third embodiment example that addresses the problem of noise in the avascular region (the third problem). In addition, the denoising processor 1300 may also perform the processes according to the below-mentioned second embodiment example that addresses the problem of dropout of thin blood vessels (the second problem).

In the case where the denoising processor 1300 is capable of performing the plurality of processes, the denoising processor 1300 may be configured to select at least one process from the plurality of processes based on any one or both of an eye fixation position and a scan area that are applied to generate the OCTA image. In other words, the denoising processor 1300 may be configured to select at least one process from the plurality of processes based on the site of the fundus Ef depicted in the OCTA image. The denoising processor 1300 may be configured to generate the denoised image by applying each process selected from the plurality of processes to the projection image generated by the image projection processor 1100.

For example, in the case where an OCTA image is generated by performing OCTA under the condition that the eye fixation position and/or the scan area are/is set to the optic nerve head, the denoising processor 1300 may perform the processes according to the below-mentioned first embodiment example that addresses the problem of irregularity in brightness in the images of thick blood vessels (the first problem). In the case where an OCTA image is generated by performing OCTA under the condition that the eye fixation position and/or the scan area are/is set to the macula, the denoising processor 1300 may perform the processes according to the below-mentioned third embodiment example that addresses the problem of noise in the avascular region (the third problem). In these cases, the denoising processor 1300 may also perform the processes according to the below-mentioned second embodiment example that addresses the problem of dropout of thin blood vessels (the second problem) in addition to the processes according to the below-mentioned first or third embodiment example.

As another example, in the case where an OCTA image is generated by performing OCTA under the condition that the eye fixation position and/or the scan area are/is set to the fundus center, which is the position between the optic nerve head and the macula, the denoising processor 1300 may perform the processes according to both the below-mentioned first embodiment example that addresses the problem of irregularity in brightness in the images of thick blood vessels (the first problem) and the processes according to the below-mentioned third embodiment example that addresses the problem of noise in the avascular region (the third problem). In addition, the denoising processor 1300 may also perform the processes according to the below-mentioned second embodiment example that addresses the problem of dropout of thin blood vessels (the second problem).

In the case where the denoising processor 1300 is capable of performing the plurality of processes, the denoising processor 1300 may be configured to select at least one process from the plurality of processes based on the field of view of the OCTA image acquired by the image acquisition unit 1000 and any one or both of the eye fixation position and scan area that are applied to generate the OCTA image. The denoising processor 1300 may be configured to generate the denoised image by applying each process selected from the plurality of processes to the projection image generated by the image projection processor 1100.

For example, in the case where an OCTA image is generated by performing OCTA under the condition that a wide field of view in which the eye fixation position and/or the scan area are/is set to the fundus center, the denoising processor 1300 may perform all of the following processes: the processes according to the below-mentioned first embodiment example that addresses the problem of irregularity in brightness in the images of thick blood vessels (the first problem), the processes according to the below-mentioned second embodiment example that addresses the problem of dropout of thin blood vessels (the second problem), and the processes according to the below-mentioned third embodiment example that addresses the problem of noise in the avascular region (the third problem).

### Image Compositing Processor 1400

The image compositing processor 1400 is configured to apply an image compositing process to the following images: the projection image generated by the image projection processor 1100, the blood vessel enhanced image generated by the blood vessel enhancement processor 1200, and the denoised image generated by the denoising processor 1300. The image generated by the image compositing process is referred to as a composite image.

The type of the image compositing process performed by the image compositing processor 1400 may be freely selected or determined. Alpha blending is an example of the image compositing process.

### Operation of Ophthalmic Apparatus

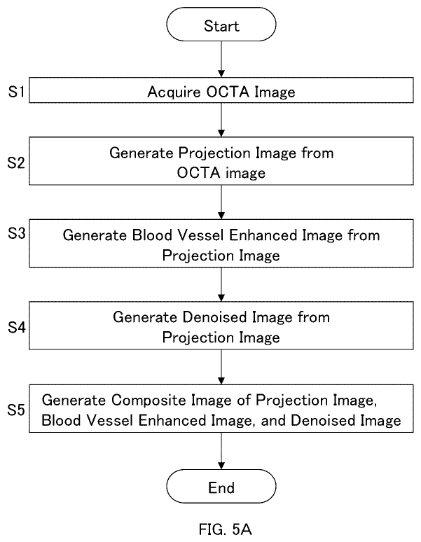

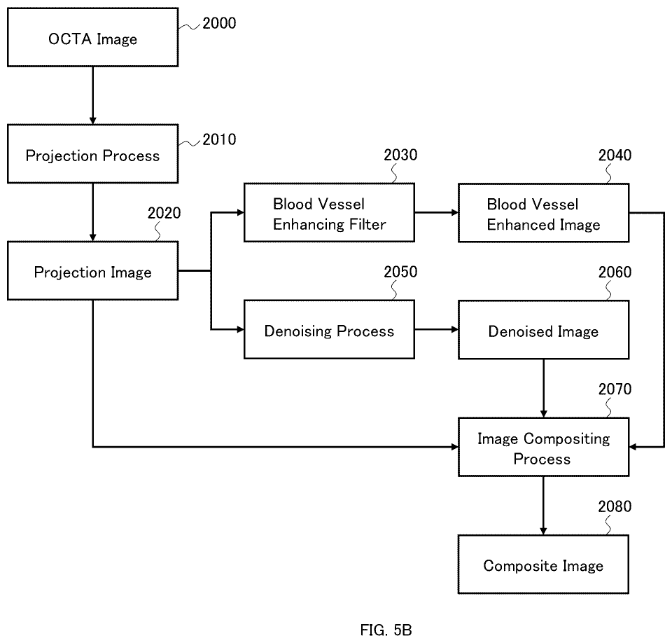

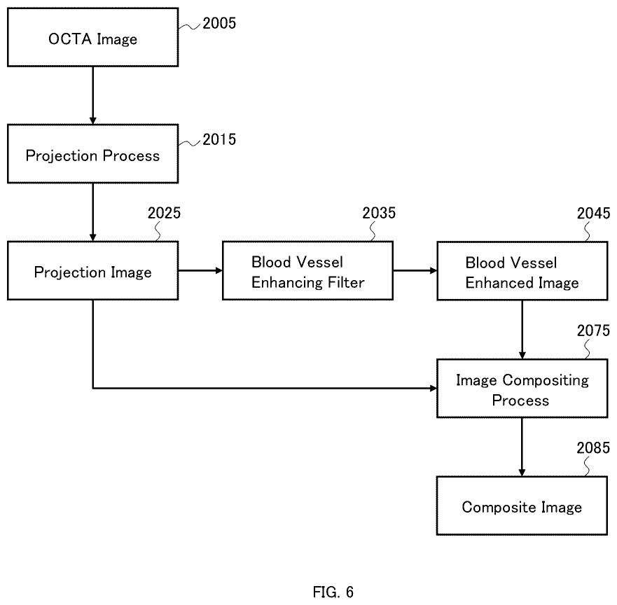

The operation of the ophthalmic apparatus 1 will be described with reference to FIGS. 5A and 5B. In addition, the differences between the operation example shown in FIGS. 5A and 5B and the comparative example shown in FIG. 6 will be described. It should be noted that the comparative example shown in FIG. 6 is not presented as a conventional technique. The comparative example of FIG. 6 is described to facilitate understanding of the operation example of FIGS. 5A and 5B.

In the operation example illustrated in FIGS. 5A and 5B, the ophthalmic apparatus 1 first acquires the OCTA image 2000 of the fundus Ef of the subject's eye E using the image acquisition unit 1000 (S1). The OCTA image 2000 acquired in the step S1 is stored in the memory 212 by the main controller 211.

Next, the ophthalmic apparatus 1 uses the image projection processor 1100 to generate the projection image 2020 by applying the projection process 2010 to the OCTA image 2000 (S2). The projection image 2020 generated in the step S2 is stored in the memory 212 by the main controller 211. The projection technique used in the projection process 2010 may be, for example, MIP, AIP, or other projection techniques.

Then, the ophthalmic apparatus 1 uses the blood vessel enhancement processor 1200 to apply the blood vessel enhancing filter 2030 configured to enhance blood vessel images to the projection image 2020, thereby generating the blood vessel enhanced image 2040 (S3). The blood vessel enhanced image 2040 generated in the step S3 is stored in the memory 212 by the main controller 211. The blood vessel enhancing filter 2030 may be, for example, a multiscale Frangi filter. The blood vessel enhancing filter 2030 may cause problems such as irregularity in brightness in the images of thick blood vessels, dropout of thin blood vessels, and noise in the avascular region.

Furthermore, by using the denoising processor 1300, the ophthalmic apparatus 1 applies the denoising process 2050 to the projection image 2020 to generate the denoised image 2060 (S4). The denoised image 2060 generated in the step S4 is stored in the memory 212 by the main controller 211.

While the present operation example is designed to perform the generation of the denoised image (S4) after the generation of the blood vessel enhanced image (S3), another operation example may be designed to perform the generation of the blood vessel enhanced image after the generation of the denoised image. In yet another operation example, the generation of the blood vessel enhanced image and the generation of the denoised image may be performed in parallel, at least in part. In other words, at least part of the generation of the blood vessel enhanced image and at least part of the generation of the denoised image may be performed simultaneously.

Subsequently, using the image compositing processor 1400, the ophthalmic apparatus 1 applies the image compositing process 2070 to the projection image 2020, the blood vessel enhanced image 2040, and the denoised image 2060, thereby generating the composite image 2080 (S5). The composite image 2080 generated in the step S5 is stored in the memory 212 by the main controller 211. The image compositing process 2070 may be alpha blending.

Conventionally, it has been common practice to display a blood vessel enhanced image generated by applying a blood vessel enhancing filter to an OCTA image. The image compositing process 2070 of the present operation example is designed to compose the projection image 2020 and the denoised image 2060 with the blood vessel enhanced image 2040. One of the reasons for composing the denoised image 2060 is to eliminate one or more problems that have occurred in the blood vessel enhanced image 2040. The problems are one or more of the following: irregularity in brightness in the images of thick blood vessels, dropout of thin blood vessels, noise in the avascular region, and like issues. On the other hand, one of the reasons for composing the projection image 2020 is to prevent the undesirable situation in which the texture of the composite image 2080 significantly differs from the texture of the original image (i.e., the OCTA image 2000, the projection image 2020) to which neither the blood vessel enhancing filter 2030 nor the denoising process 2050 has been applied. The advantageous effects described above are characteristic and significant features unique to the image compositing process 2070 in the present embodiment example. This concludes the explanation of the present operation example (End).

Several non-limiting examples of processes that can be performed based on the images (and various types of information associated with these images) handled in the present operation example are described below.

The ophthalmic apparatus 1 may be configured to be able to display the composite image 2080 by using the main controller 211 and the display unit 241. The ophthalmic apparatus 1 may be configured to be able to display any one or more of the following images: the OCTA image 2000, the projection image 2020, the blood vessel enhanced image 2040, and the denoised image 2060. The ophthalmic apparatus 1 may further be configured to be able to display a fundus image or an anterior segment image acquired by the fundus camera unit 2. The user may conduct medical image interpretation and medical report creation by referring to the images displayed.

The ophthalmic apparatus 1 may be configured to be able to apply processing to any one or more of the following images by using either or both of the image constructing processor 220 and the data processor 230: the OCTA image 2000, the projection image 2020, the blood vessel enhanced image 2040, the denoised image 2060, the composite image 2080, the fundus image, and the anterior segment image. This processing may include image processing and/or signal processing. Non-limiting examples of the processing include image analysis, image evaluation, image quality improvement, segmentation, rendering, and other processes.

The processing performed by either or both of the image constructing processor 220 and the data processor 230 may include processing executed by using a model constructed by machine learning. Non-limiting examples of the machine learning model may be configured to be able to perform any of the following processes: image interpretation and report generation of any one or more images (e.g., the composite image 2080) handled by the ophthalmic apparatus 1; comparison of two or more images (e.g., the OCTA image 2020 and the composite image 2080); generation of training data for further machine learning (e.g., updating of training data set).

The ophthalmic apparatus 1 may be configured to be able to transmit any one or more of the following images to an external device by using the communication interface included in the arithmetic and control unit 200: the OCTA image 2000, the projection image 2020, the blood vessel enhanced image 2040, the denoised image 2060, the composite image 2080, the fundus image, and the anterior segment image.

The ophthalmic apparatus 1 may be configured to be able to store one or more of the following images into a recording medium by using the drive device included in the arithmetic and control unit 200: the OCTA image 2000, the projection image 2020, the blood vessel enhanced image 2040, the denoised image 2060, the composite image 2080, the fundus image, and the anterior segment image.

Next, some differences between the operation example shown in FIGS. 5A and 5B and the comparative example shown in FIG. 6 will be described, along with several non-limiting features of the present operation example.

The ophthalmic apparatus of the comparative example shown in FIG. 6 executes the following series of processes: acquisition of the OCTA image 2005; generation of the projection image 2025 by applying the projection process 2015 to the OCTA image 2005; generation of the blood vessel enhanced image 2045 by applying the blood vessel enhancing filter 2035 to the projection image 2025; and generation of the composite image 2085 by applying the image compositing process 2075 to the projection image 2025 and the blood vessel enhanced image 2045.

Comparing the present operation example shown in FIGS. 5A and 5B with the comparative example shown in FIG. 6, the present operation example differs from the comparative example in the following points: First, the present operation example performs the process of generating the denoised image 2060 from the projection image 2020 (i.e., the denoising process 2050). Second, the present operation example composes, in the image compositing process 2070, not only the projection image 2020 and the blood vessel enhanced image 2040 but also the denoised image 2060.

In the comparative example, the projection image and the blood vessel enhanced image are composed without performing a process corresponding to the denoising process 2050 of the present operation example. Therefore, the resulting composite image still includes undesirable conditions caused by the blood vessel enhancing filter 2035 such as irregularity in brightness in the images of thick blood vessels, dropout of the image of thin blood vessels, and noise in the FAZ. These undesirable conditions lead to degradation in the quality of blood vessel representation (blood vessel visualization, blood vessel images) of eye fundus. In contrast, according to the present operation example, such undesirable conditions can be eliminated or reduced, thereby enabling the provision of a high-quality fundus blood vessel image (i.e., an OCTA image with enhanced blood vessels). In addition, the present operation example enables the generation of a fundus blood vessel image having texture that is closer to that of the original OCTA image 2000 or the projection image 2020.

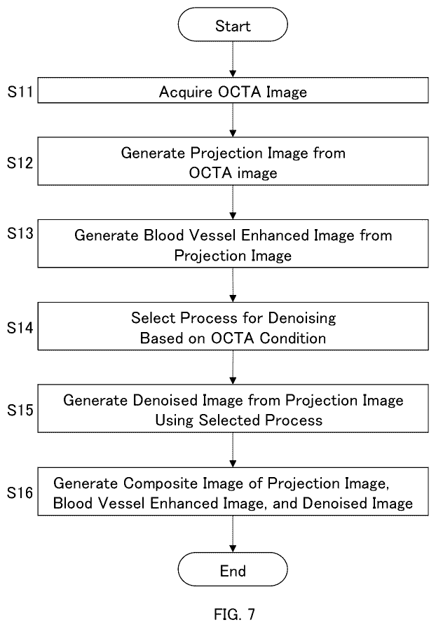

Another operation example of the ophthalmic apparatus 1 is shown in FIG. 7. In the present operation example, similar to the steps S1 to S3 shown in FIG. 5A, the ophthalmic apparatus 1 acquires an OCTA image of the fundus Ef of the subject's eye E (S11), generates a projection image by applying a projection process to the OCTA image (S12), and applies a blood vessel enhancing filter used for blood vessel image enhancement to the projection image, thereby generating a blood vessel enhanced image (S13).

Furthermore, the ophthalmic apparatus 1 selects one or more processes from a plurality of processes prepared for denoising, based on a condition of OCTA applied to the fundus Ef to generate the OCTA image acquired in the step S11, for example, by using the main controller 211 or the denoising processor 1300 (S14). The type of the process selected in the step S14 is stored in the memory 212 by the main controller 211.

The plurality of processes prepared for denoising may include, in some examples, the processes according to the three embodiment examples described below. More specifically, the plurality of processes in the present operation example may include any of the following: one or more processes according to the first embodiment example that addresses the problem of irregularity in brightness in the images of thick blood vessels; one or more processes according to the second embodiment example that addresses the problem of dropout of thin blood vessels; and one or more processes according to the third embodiment example that addresses the problem of noise in the avascular region.

The OCTA condition may include, for example, one or more of the following conditions: the field of view, the eye fixation position, and the scan area. Information on the OCTA condition is obtained together with the OCTA image in the step S11. The form of the information indicating the OCTA condition may be freely selected or determined. As non-limiting examples, the OCTA condition information may be information recorded as supplementary information of the OCTA image (e.g., a DICOM tag, or information of other forms), information recorded in the subject's information (e.g., an electronic medical record, an image interpretation report, or information of other forms), or information generated by applying an analysis process to the OCTA image or an image generated therefrom (e.g., the projection image, the blood vessel enhanced image, or images of other forms).

The ophthalmic apparatus 1, using the denoising processor 1300, applies a denoising process including the process selected in the step S14 to the projection image generated in the step S12, thereby generating a denoised image (S15). In this operation example as well, the order of the generation of the blood vessel enhanced image and the generation of the denoised image may be freely determined.

Subsequently, using the image compositing processor 1400, the ophthalmic apparatus 1 applies an image compositing process to the following images, thereby generating a composite image: the projection image generated in the step S12, the blood vessel enhanced image generated in the step S13, and the denoised image generated in the step S15 (S16). This concludes the explanation of the present operation example (End).

According to the operation example of FIG. 7, in addition to achieving the same advantageous effects as those of the operation example shown in FIGS. 5A and 5B, it is also possible to perform a denoising process using the process selected in accordance with the OCTA condition. Due to the latter operation that is specific to the present operation example, the denoising process can be performed with unnecessary or less relevant processes excluded and with one or more appropriate processes selected in accordance with the OCTA condition. Accordingly, the denoising process can be performed effectively and efficiently.

### First Embodiment Example

The first embodiment example gives a description of several aspect examples of the processes for addressing the first problem, which is the problem of irregularity in brightness in the images of thick blood vessels, caused by the use of a blood vessel enhancing filter.

A blood vessel enhancing filter, such as a multiscale Frangi filter, is configured to extract blood vessels of various thicknesses. More specifically, the blood vessel enhancing filter is configured to vary the value of a predetermined scale parameter to respectively detect blood vessels of various thicknesses. Although the problem of irregularity in brightness in the images of thick blood vessels may be (substantially) resolved by using a scale parameter corresponding to the thick blood vessels, this may give rise to other problems such as blurring of blood vessel images, two separate blood vessels being depicted as a single connected vessel, or other types of problems. The first embodiment example addresses these additional problems while also eliminating the problem of irregularity in brightness in the images of thick blood vessels.

In the first embodiment example, the denoising processor 1300 is configured to apply a blood vessel image extracting process configured to extract blood vessel images of widths belonging to the first range, to a projection image generated from the OCTA image by the image projection processor 1100.

The first range relating to the widths of blood vessels to be extracted may be determined in advance, or may be determined based on an OCTA image or an image generated therefrom (e.g., the projection image). In the former case (predetermination of the first range), the first range may be determined based on a standard value of blood vessel diameter. In some aspect examples, a plurality of standard values corresponding to a plurality of conditions are prepared in advance based on patient attributes (such as age, sex, or race) and/or imaging conditions (such as the site of eye fundus), and a standard value is selectively used, as the first range, in accordance with the image to which the blood vessel image extracting process is applied. In the latter case (determination of the first range on the basis of an image), the ophthalmic apparatus 1 (e.g., the denoising processor 1300) may be configured to analyze the OCTA image or the projection image to derive a distribution or a statistical value of blood vessel diameters, and to determine the first range based on the distribution or the statistical value.

Furthermore, the denoising processor 1300 is configured to apply an erosion process to the image generated through the blood vessel image extracting process, thereby generating an eroded image in which a reduced blood vessel image is depicted. The reduced blood vessel image corresponds to the extracted blood vessel image, which is the blood vessel image extracted by the blood vessel image extracting process, having the width reduced by the erosion process.

In a wider sense, an erosion process is a type of morphological processing (morphological transformations, morphological operations) that reduces a region of interest by replacing the value of a pixel in the region of interest with a value of adjacent pixels. In the first embodiment example, the erosion process acts to reduce the dimension of the blood vessel image (i.e., the region of interest) depicted in the image generated through the blood vessel image extracting process. As a result, an eroded image is generated in which a blood vessel image (reduced blood vessel image), having a smaller width than the width of the blood vessel image extracted by the blood vessel image extracting process, is depicted.

The blood vessel enhancement processor 1200 of the present embodiment example is configured to generate the first blood vessel enhanced image by applying a multiscale Frangi filter to the projection image generated from the OCTA image by the image projection processor 1100.

The blood vessel enhancement processor 1200 is further configured to apply a Frangi filter of a scale corresponding to the second range, which is designed to be smaller than the first range used in the blood vessel image extracting process, to the projection image generated from the OCTA image by the image projection processor 1100.

In addition, the blood vessel enhancement processor 1200 is configured to apply gamma correction that increases brightness of a blood vessel image to the image generated by applying the Frangi filter of the scale corresponding to the second range to the projection image, thereby generating the second blood vessel enhanced image.

The denoising processor 1300 of the present embodiment example is configured to generate a denoised image based on the eroded image, the first blood vessel enhanced image, and the second blood vessel enhanced image. Here, the eroded image is the image in which the reduced blood vessel image, having the width smaller than that of the blood vessel image extracted through the blood vessel image extracting process, is depicted. The first blood vessel enhanced image is the image generated by applying the multiscale Frangi filter to the projection image. The second blood vessel enhanced image is the image generated by applying, to the projection image, both the Frangi filter of the scale corresponding to the second range smaller than the first range of the blood vessel image extracting process, and the gamma correction.

The image compositing processor 1400 of the present embodiment example is configured to generate a composite image by applying the image compositing process to the denoised image, the projection image, and the first blood vessel enhanced image. The denoised image here is the image generated from the eroded image, the first blood vessel enhanced image, and the second blood vessel enhanced image. The image compositing process may be performed using alpha blending or another image compositing method.

The first embodiment example is capable of generating a blood vessel enhanced OCTA image in which no or less irregularity in brightness appears in the images of thick blood vessels, by performing the series of processes implemented by employing the above-described configuration. The mechanism by which such an advantageous effect is achieved will be described after explanation of several specific examples.

First, one specific example (referred to as the first specific example) of the denoised image generation process in the first embodiment example will be described.

In the first specific example of the first embodiment example, the denoising processor 1300 is configured to analyze the first blood vessel enhanced image to identify the first sub-image of the first blood vessel enhanced image. The first sub-image corresponds to the reduced blood vessel image in the eroded image. This process may employ, for example, a thresholding process with respect to brightness values, a segmentation method, a segmentation method using a machine learning model, or other processes freely selected or determined.

Similarly, the denoising processor 1300 is configured to analyze the second blood vessel enhanced image to identify the second sub-image of the second blood vessel enhanced image. The second sub-image corresponds to the reduced blood vessel image in the eroded image.

Furthermore, the denoising processor 1300 is configured to generate the denoised image by selecting a higher brightness value between a brightness value of a pixel of the first sub-image of the first blood vessel enhanced image and a brightness value of a corresponding pixel of the second sub-image of the second blood vessel enhanced image.

Since the first blood vessel enhanced image and the second blood vessel enhanced image are both generated from the same OCTA image, a natural correspondence can be defined or established between the group of pixels constituting the first blood vessel enhanced image and the group of pixels constituting the second blood vessel enhanced image. By using this correspondence, it is possible to identify, for any pixel in the first blood vessel enhanced image, a corresponding pixel in the second sub-image that corresponds to the pixel in the first sub-image.

For each pair of corresponding pixels between the first sub-image and the second sub-image that have been associated in this manner, the denoising processor 1300 compares the brightness values of the pair of pixels, and identifies the pixel with the higher brightness value among the pair of pixels. The denoised image is generated by using a group of pixels identified by performing this process for each pixel pair.

The image compositing processor 1400 is configured to generate the composite image by applying the image compositing process to the following images: the denoised image generated in this manner from the first sub-image and the second sub-image, the projection image, and the first blood vessel enhanced image.

Next, a further specific example (referred to as the second specific example) of the first specific example of the first embodiment example will be described. The second specific example of the first embodiment example provides a further detailed implementation of the denoising process described in the first specific example.

In the second specific example of the first embodiment example, the denoising processor 1300 is configured to determine the first sub-image of the first blood vessel enhanced image by applying a masking process based on the reduced blood vessel image in the eroded image to the first blood vessel enhanced image.

Furthermore, the denoising processor 1300 is configured to determine the second sub-image of the second blood vessel enhanced image by applying the masking process based on the reduced blood vessel image in the eroded image to the second blood vessel enhanced image.

The masking process applied to the first blood vessel enhanced image and the masking process applied to the second blood vessel enhanced image may be the same process. For example, in the masking process for the first blood vessel enhanced image, the denoising processor 1300 may apply a mask to the image region of the first blood vessel enhanced image that corresponds to the reduced blood vessel image in the eroded image. Similarly, in the masking process for the second blood vessel enhanced image, the denoising processor 1300 may apply a mask to the image region of the second blood vessel enhanced image that corresponds to the reduced blood vessel image in the eroded image. As a result, the first sub-image is identified from the first blood vessel enhanced image, and the second sub-image is identified from the second blood vessel enhanced image.

Next, a further specific example (referred to as the third specific example) of the first or second specific example of the first embodiment example will be described. In the third specific example of the first embodiment example, the image projection processor 1100 may be configured to perform MIP as the projection process for the OCTA image, thereby generating the projection image (MIP image). MIP is an image projection method that is designed to select and project the maximum value from among the brightness values of a group of pixels arranged along the projection direction.

Next, a further specific example (referred to as the fourth specific example) of any of the first to third specific examples of the first embodiment example will be described. In the fourth specific example of the first embodiment example, the denoising processor 1300 may be configured to perform Otsu's method as the blood vessel image extracting process applied to the projection image to extract the blood vessel image of the width belonging to the first range.

In general, Otsu's method is an algorithm for binarizing a brightness image, and is particularly effective for binarizing an image whose brightness histogram has two peaks (i.e., an image having a bimodal histogram). The Otsu's method algorithm is configured to search for a threshold that minimizes the intra-class variance, which is defined as the weighted sum of the variances of two classes. In other words, the Otsu's method algorithm is configured to find a threshold that lies between the two peaks of the bimodal histogram, that is, to find a threshold that minimizes the intra-class variance of the two classes classified by the binarization.

In the fourth specific example of the first embodiment example, Otsu's method can be used to selectively extract the image of a relatively thick blood vessel (i.e., the blood vessel image of the width belonging to the first range) from the images of blood vessels of various thicknesses depicted in the projection image. The denoising processor 1300 may be configured to apply the erosion process to the image of thick blood vessel extracted using Otsu's method, thereby generating the eroded image in which a reduced blood vessel image of the thick blood vessel image is depicted.

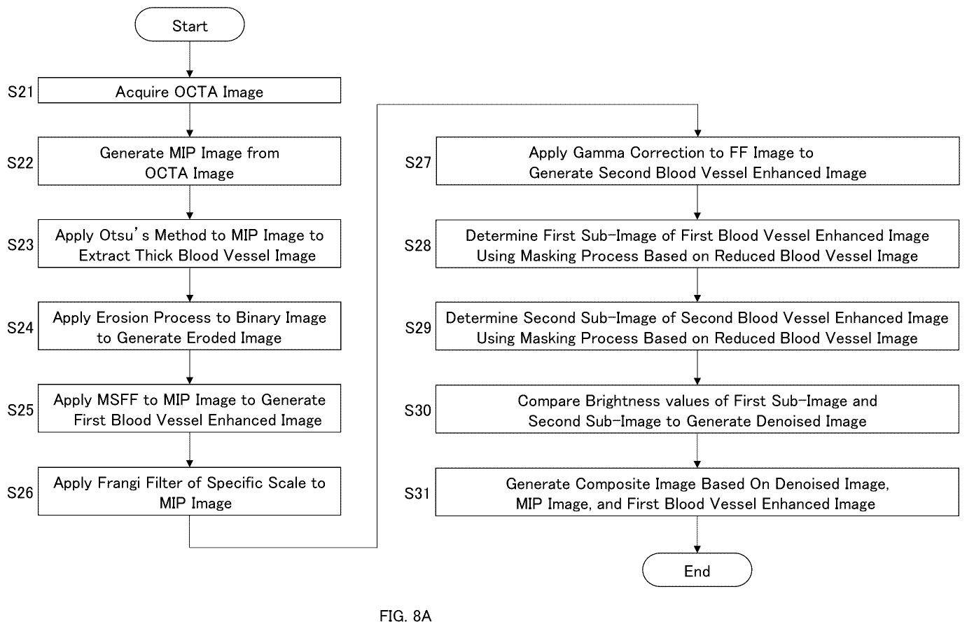

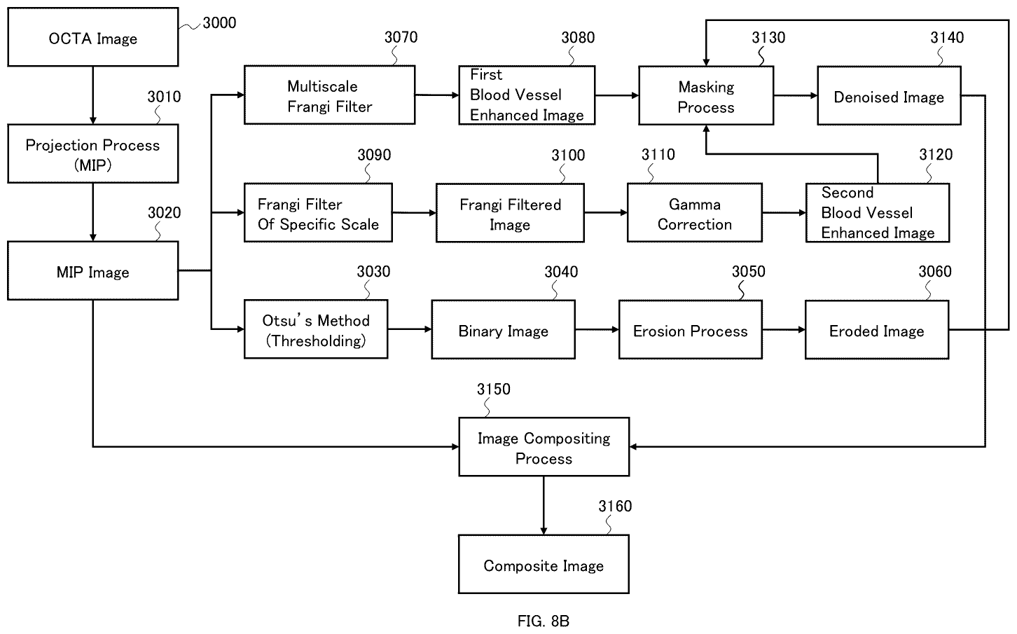

Subsequently, the operation of the ophthalmic apparatus 1 of the first embodiment example will be described with reference to FIGS. 8A and 8B. The operation example shown in FIGS. 8A and 8B includes the processes according to the first to fourth specific examples described above.

In the operation example shown in FIGS. 8A and 8B, the ophthalmic apparatus 1 first acquires the OCTA image 3000 of the fundus Ef of the subject's eye E by the use of the image acquisition unit 1000 (S21). The acquired OCTA image 3000 is stored in the memory 212 by the main controller 211.

The ophthalmic apparatus 1, using the image projection processor 1100, then applies the MIP 3010 to the OCTA image 3000 to generate the MIP image 3020 (S22). The generated MIP image 3020 is stored in the memory 212 by the main controller 211.

After the step S22, the following three process lines are performed: the processes of the steps S23 and S24 (corresponding to the reference characters 3030 to 3060 shown in FIG. 8B), the processes of the step S25 (corresponding to the reference characters 3070 and 3080 shown in FIG. 8B), and the processes of the steps S26 and S27 (corresponding to the reference characters 3090 to 3120 shown in FIG. 8B). The order in which these three process lines are performed may be freely determined. Two or more of the three process lines may be performed in parallel, at least in part.

The ophthalmic apparatus 1, using the denoising processor 1300, applies the Otsu's method 3030, which corresponds to the blood vessel image extracting process for extracting a blood vessel image of a width belonging to the first range, to the MIP image 3020, thereby generating the binary image 3040 in which the image of a thick blood vessel is selectively depicted (S23). The generated binary image 3040 is stored in the memory 212 by the main controller 211.

Furthermore, the ophthalmic apparatus 1 applies the erosion process 3050 to the binary image 3040 by the use of the denoising processor 1300 to reduce the width of the thick blood vessel image depicted in the binary image 3040 (S24). In the eroded image 3060 generated from the binary image 3040 by the erosion process 3050, the reduced blood vessel image, which is the blood vessel image obtained by reducing the width of the thick blood vessel image, is depicted. The generated eroded image 3060 is stored in the memory 212 by the main controller 211.

Using the blood vessel enhancement processor 1200, the ophthalmic apparatus 1 applies the multiscale Frangi filter 3070 to the MIP image 3020, thereby generating the first blood vessel enhanced image 3080 (S25). The generated first blood vessel enhanced image 3080 is stored in the memory 212 by the main controller 211.

In addition, using the blood vessel enhancement processor 1200, the ophthalmic apparatus 1 applies the Frangi filter 3090, whose scale corresponds to the second range that is smaller than the first range of the blood vessel image extracting process (the Otsu's method 3030) of the step S23, to the MIP image 3020, thereby generating the Frangi filtered image 3100 (S26). The generated Frangi filtered image 3100 is stored in the memory 212 by the main controller 211.

Furthermore, by the use of the blood vessel enhancement processor 1200, the ophthalmic apparatus 1 applies the gamma correction 3110 for increasing the brightness of the blood vessel image to the Frangi filtered image 3100, thereby generating the second blood vessel enhanced image 3120 (S27). The generated second blood vessel enhanced image 3120 is stored in the memory 212 by the main controller 211.

Thereafter, using the denoising processor 1300, the ophthalmic apparatus 1 applies the masking process 3130 based on the reduced blood vessel image depicted in the eroded image 3060 to the first blood vessel enhanced image 3080 and the second blood vessel enhanced image 3120 (S28 and S29).

Specifically, employing the denoising processor 1300, the ophthalmic apparatus 1 determines the first sub-image of the first blood vessel enhanced image 3080 by applying the masking process 3130 to the first blood vessel enhanced image 3080 (S28). The first sub-image or the coordinate information in the first blood vessel enhanced image 3080 corresponding to the first sub-image is stored in the memory 212 by the main controller 211.

Similarly, by the use of the denoising processor 1300, the ophthalmic apparatus 1 determines the second sub-image of the second blood vessel enhanced image 3120 by applying the masking process 3130 to the second blood vessel enhanced image 3120 (S29). The second sub-image or the coordinate information in the second blood vessel enhanced image 3120 corresponding to the second sub-image is stored in the memory 212 by the main controller 211.

In a subsequent step, using the denoising processor 1300, the ophthalmic apparatus 1 compares the brightness values of the group of pixels constituting the first sub-image identified from the first blood vessel enhanced image 3080 in the step S28 with the brightness values of the group of pixels constituting the second sub-image identified from the second blood vessel enhanced image 3120 in the step S29, thereby generating the denoised image 3140 (S30). The generated denoised image 3140 is stored in the memory 212 by the main controller 211.

In some specific implementations, by using the denoising processor 1300, the ophthalmic apparatus 1 generates the denoised image 3140 by selecting the higher brightness value between the brightness value of a pixel of the first sub-image of the first blood vessel enhanced image 3080 and the brightness value of the corresponding pixel of the second sub-image of the second blood vessel enhanced image 3120. In this manner, the present operation example is designed to address the problem that a region near the centerline of the image of the thick blood vessel is represented with low brightness by increasing the brightness in that region.

Next, by the use of the image compositing processor 1400, the ophthalmic apparatus 1 generates the composite image 3160 by applying the image compositing process 3150 to the denoised image 3140, the MIP image 3020, and the first blood vessel enhanced image 3080 (S31). The generated composite image 3160 is stored in the memory 212 by the main controller 211. This concludes the explanation of the present operation example (End).

The ophthalmic apparatus 1 may be configured to display at least one of the following images: the OCTA image 3000, the MIP image 3020, the binary image 3040, the eroded image 3060, the first blood vessel enhanced image 3080, the second blood vessel enhanced image 3120, the denoised image 3140, and the composite image 3160. Further, the ophthalmic apparatus 1 may be configured to apply a freely selected or designed process to at least one of these images. In addition, the ophthalmic apparatus 1 may be configured to transmit at least one of these images to an external device, and/or record at least one of these images on a recording medium.

The ophthalmic apparatus 1 according to the first embodiment example is configured to apply the multiscale Frangi filter to the projection image of the OCTA image to generate the first blood vessel enhanced image in which the images of blood vessels of various thicknesses are depicted. Along with this, the ophthalmic apparatus 1 according to the first embodiment example is configured to extract a blood vessel image from the same projection image using the Frangi filter of an appropriate scale, which is designed to extract the image of a blood vessel with a width that belongs to the second range, thereby generating the Frangi filtered image. Here, the second range is smaller than the first range corresponding to the width of the thick blood vessel.

Since this Frangi filtered image is generated using the Frangi filter of the scale corresponding to the second range, the density of the blood vessel image depicted in the Frangi filtered image is lower than the density of the thick blood vessel image to be extracted. Accordingly, the first embodiment example applies the gamma correction to the Frangi filtered image in order to increase the density of the blood vessel image depicted in the Frangi filtered image.

Furthermore, in the first embodiment example, the denoised image is generated based on the Frangi filtered image (i.e., the second blood vessel enhanced image) in which the density (i.e., brightness, contrast) of the blood vessel image has been enhanced by the gamma correction. The denoised image thus generated is then composed with the first blood vessel enhanced image generated by applying the multiscale Frangi filter to the OCTA image. The denoised image is generated using the masking process. This masking process makes it possible to apply subsequent processes to an appropriate blood vessel region.

According to the first embodiment example described thus far, it is possible to eliminate or reduce the problem of irregularity in brightness occurring in images of thick blood vessels. In addition, problems that may arise when using a Frangi filter of a scale corresponding to a larger blood vessel diameter (such as the problem of the blood vessel image being represented in a blurred state or a problem of two separate blood vessels being depicted as connected) do not occur.

In addition, since the first embodiment example is configured to compose not only the first blood vessel enhanced image and the denoised image but also the projection image, a composite image with a texture close to that of the original image can be generated.

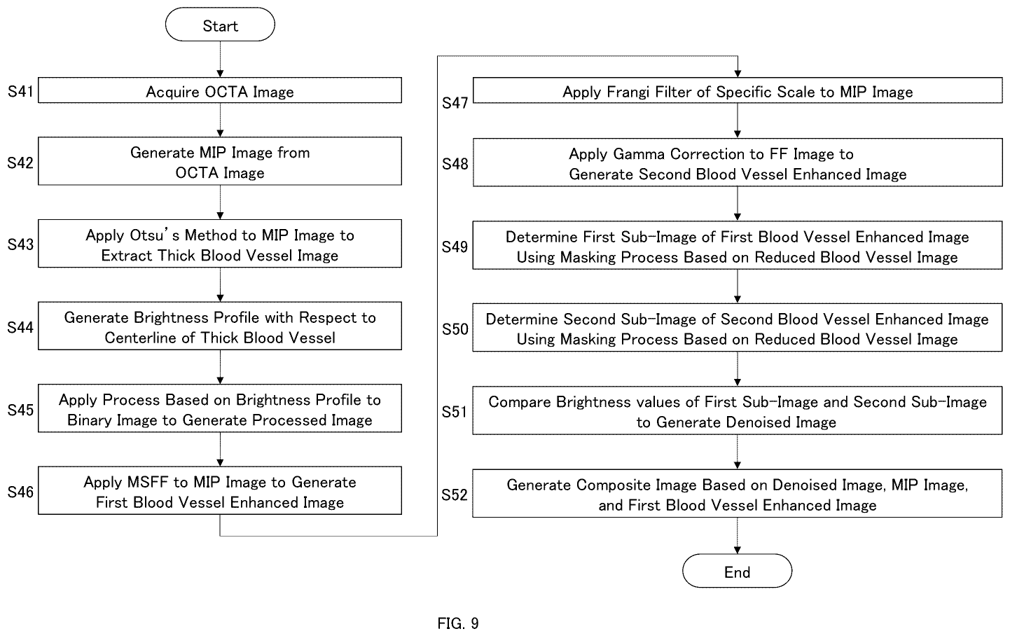

Another operation example of the ophthalmic apparatus 1 is shown in FIG. 9. In the present operation example, a brightness profile (brightness map) related to the distance from the centerline of the blood vessel image is used instead of the erosion process described above. Employing the brightness profile increases the brightness of the region in the vicinity of the centerline of the blood vessel image, thereby addressing the irregularity in brightness in the images of thick blood vessels.

In the operation example shown in FIG. 9, similar to the steps S21 to S23 shown in FIG. 8A, the ophthalmic apparatus 1 acquires an OCTA image of the fundus Ef of the subject's eye E (S41), applies MIP to this OCTA image to generate an MIP image (S42), and applies Otsu's method to this MIP image to generate a binary image in which the image of a thick blood vessel is depicted (S43). The generated binary image is stored in the memory 212 by the main controller 211.

Following that, using the denoising processor 1300, the ophthalmic apparatus 1 analyzes the thick blood vessel image extracted in the binary image generated in the step S43 to determine the centerline of the thick blood vessel image, and determines a brightness profile with respect to the centerline (S44). The process of determining the centerline of the thick blood vessel image may include, for example, a thinning process or a skeletonization process. The brightness profile with respect to the centerline of the thick blood vessel image is information that associates a distance from the centerline with a brightness value, and is a map that represents the distribution of brightness values in the thick blood vessel image on the basis of the location of the centerline. It should be noted that possible methods of representing such a brightness profile in a thick blood vessel image are not limited to the aspect of the present example. A brightness profile representation method may be freely selected or freely determined.

Next, the ophthalmic apparatus 1 applies a process based on the brightness profile generated in the step S44 to the binary image generated in the step S43 by the use of the denoising processor 1300 (S45). The image generated in the step S45 is referred to as a processed image. The processed image is stored in the memory 212 by the main controller 211.

Similar to the eroded image described above, a reduced blood vessel image in which the width of the thick blood vessel image is reduced, is depicted in the processed image. Expressed another way, the process based on the brightness profile applied to the binary image in the step S45 is configured to reduce the width of the thick blood vessel image. In some examples, this process may be configured to identify, based on the brightness profile, a region with relatively low brightness in the vicinity of the centerline of the thick blood vessel.

Moreover, by employing the blood vessel enhancement processor 1200, the ophthalmic apparatus 1 applies a multiscale Frangi filter to the MIP image generated in the step S42 to generate the first blood vessel enhanced image (S46). The blood vessel enhanced image generated is stored in the memory 212 by the main controller 211.

Furthermore, by employing the blood vessel enhancement processor 1200, the ophthalmic apparatus 1 applies a Frangi filter of a scale corresponding to the second range smaller than the first range in the blood vessel image extracting process (Otsu's method) of the step S23 to the MIP image generated in the step S42, thereby generating a Frangi filtered image (S47). The Frangi filtered image generated is stored in the memory 212 by the main controller 211.

In addition, by the use of the blood vessel enhancement processor 1200, the ophthalmic apparatus 1 applies gamma correction that increases the brightness of the blood vessel image to the Frangi filtered image generated in the step S47, thereby generating the second blood vessel enhanced image (S48). The second blood vessel enhanced image generated is stored in the memory 212 by the main controller 211.

Thereafter, the ophthalmic apparatus 1, using the denoising processor 1300, determines the first sub-image of the first blood vessel enhanced image and the second sub-image of the second blood vessel enhanced image, by applying the masking process based on the reduced blood vessel image depicted in the processed image generated in the step S45 to each of the first blood vessel enhanced image generated in the step S46 and the second blood vessel enhanced image generated in the step S48 (S49 and S50). The first sub-image or the coordinate information in the first blood vessel enhanced image corresponding to the first sub-image is stored in the memory 212 by the main controller 211, and the second sub-image or the coordinate information in the second blood vessel enhanced image corresponding to the second sub-image is stored in the memory 212 by the main controller 211.

Subsequently, using the denoising processor 1300, the ophthalmic apparatus 1 compares the brightness values of the group of pixels constituting the first sub-image identified from the first blood vessel enhanced image in the step S49 with the brightness values of the group of pixels constituting the second sub-image identified from the second blood vessel enhanced image in the step S50, thereby generating the denoised image (S51). The denoised image generated is stored in the memory 212 by the main controller 211.

In some specific implementations, using the denoising processor 1300, the ophthalmic apparatus 1 generates the denoised image by selecting the higher brightness value between the brightness value of a pixel of the first sub-image of the first blood vessel enhanced image and the brightness value of the corresponding pixel of the second sub-image of the second blood vessel enhanced image. In this manner, the present operation example is designed to increase the brightness in the region near the centerline of the image of the thick blood vessel, thereby addressing the problem in which that region is represented with low brightness.

Next, employing the image compositing processor 1400, the ophthalmic apparatus 1 generates the composite image by applying the image compositing process to the following images: the denoised image generated in the step S51, the MIP image generated in the step S42, and the first blood vessel enhanced image generated in the step S46 (S52). The composite image generated is stored in the memory 212 by the main controller 211. This concludes the explanation of the present operation example (End).

The ophthalmic apparatus 1 may display any of the images acquired or generated in the operation example shown in FIG. 9. The ophthalmic apparatus 1 may apply a freely selected or designed process to any of the images acquired or generated in the operation example shown in FIG. 9. The ophthalmic apparatus 1 may transmit, to an external device, any of the images acquired or generated in the operation example shown in FIG. 9. The ophthalmic apparatus 1 may record, in a recording medium, any of the images acquired or generated in the operation example shown in FIG. 9.

Similar to the operation example illustrated in FIGS. 8A and 8B, the operation example illustrated in FIG. 9 is also capable of eliminating or reducing the problem of irregularity in brightness occurring in the images of thick blood vessels. In addition, problems that may arise when using a Frangi filter of a scale corresponding to a larger blood vessel diameter (such as the problem of the blood vessel image being represented in a blurred state or a problem of two separate blood vessels being depicted as connected) do not occur. Furthermore, the operation example illustrated in FIG. 9 makes it possible to generate a composite image with a texture close to that of the original image.

### Second Embodiment Example

In the second embodiment example, a description will be given of several aspect examples of the processes for addressing the second problem (i.e., the problem of dropout of the image of thin blood vessels) caused by the use of the blood vessel enhancing filter.

The second problem that the second embodiment example deals with corresponds to the phenomenon in which the images of relatively thin blood vessels that should be clearly depicted in the blood vessel enhanced image is subject to dropout. For example, if the images of thin blood vessels that actually exist in the arcade region of the eye fundus are not visualized, there is a possibility that a disease may be suspected during image interpretation or a false positive determination may be made during analysis. Such a situation can be avoided in the second embodiment example.

In the second embodiment example, the image projection processor 1100 is configured to apply two mutually different types of projection processes to the OCTA image acquired by the image acquisition unit 1000. Stated differently, the image projection processor 1100 is configured to apply the first projection process to the OCTA image to generate the first projection image, and apply the second projection process, which is a different type of process from the first projection process, to the OCTA image to generate the second projection image.

In some aspect examples, the first projection process is MIP and the second projection process is AIP. MIP is highly effective in depicting blood vessels and is widely used in various angiographic methods, including OCTA. However, MIP has a drawback in that it is prone to noise contamination. In contrast, AIP has the characteristic of being less susceptible to noise contamination and has a noise reduction effect. In other words, in the second embodiment example, the AIP has both a function as the image projection process and a function as the denoising process.

In the second embodiment example, the blood vessel enhancement processor 1200 is configured to apply a multiscale Frangi filter to the first projection image generated from the OCTA image by the first projection process, thereby generating the first blood vessel enhanced image.

The blood vessel enhancement processor 1200 is further configured to apply a Frangi filter of a scale corresponding to the range of a width of a capillary to the second projection image generated from the OCTA image by the second projection process. The image generated from the second projection image by this Frangi filter is referred to as a Frangi filtered image.

The blood vessel enhancement processor 1200 is yet further configured to apply gamma correction that increases brightness of a blood vessel image to the Frangi filtered image, thereby generating the second blood vessel enhanced image. In an aspect in which a projection method with a noise reduction effect, such as AIP, is used in the second projection process, the second blood vessel enhanced image generated through the second projection process and the Frangi filter may be treated as a denoised image generated by the denoising processor 1300.

In the second embodiment example, the image compositing processor 1400 is configured to generate the composite image by applying the image compositing process to the following images: the first projection image generated from the OCTA image by the use of the first projection process; the first blood vessel enhanced image generated from the first projection image by the use of the multiscale Frangi filter; and the second blood vessel enhanced image generated by applying the Frangi filter and the gamma correction to the second projection image generated from the OCTA image by the use of the second projection process. The image compositing process may be performed using alpha blending or another image compositing method.

By executing a series of processes that can be implemented by such a configuration, the second embodiment example is capable of generating a blood vessel enhanced OCTA image in which dropout of the image of thin blood vessels (e.g., capillaries) does not occur.

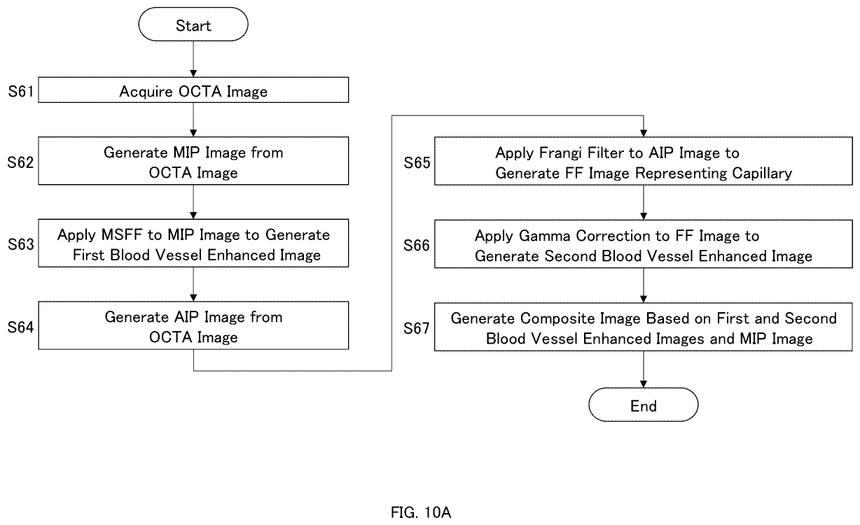

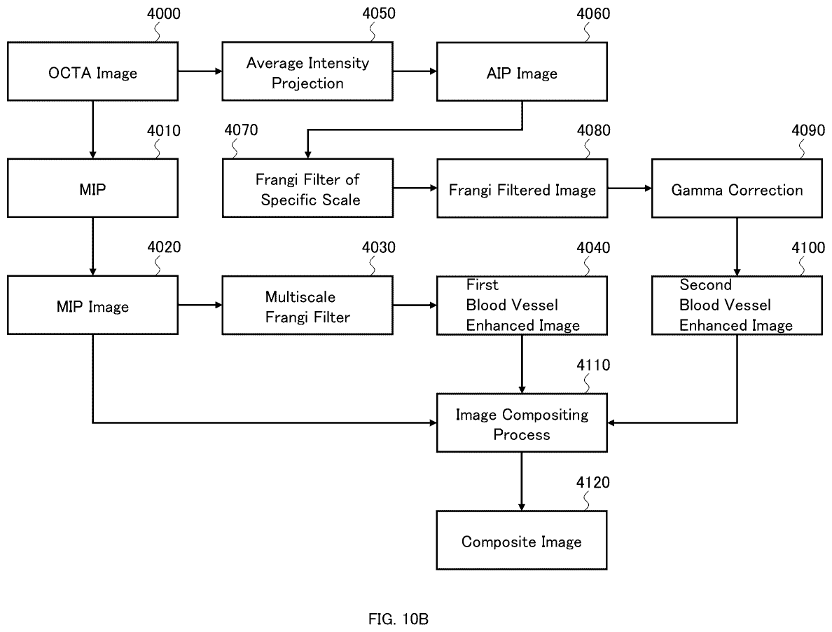

Next, the operation of the ophthalmic apparatus 1 of the second embodiment example will be described with reference to FIGS. 10A and 10B.

In the operation example shown in FIGS. 10A and 10B, the ophthalmic apparatus 1 first acquires the OCTA image 4000 of the fundus Ef of the subject's eye E by the use of the image acquisition unit 1000 (S61). The OCTA image 4000 acquired is stored in the memory 212 by the main controller 211.

After the step S61, the following two process lines are performed: the processes of the steps S62 and S63 (corresponding to the reference characters 4010 to 4040 shown in FIG. 10B), and the processes of the steps S64 to S66 (corresponding to the reference characters 4050 to 4100 shown in FIG. 10B). The order in which the two process lines are performed may be freely determined. The two process lines may be performed in parallel, at least in part.

The ophthalmic apparatus 1, using the image projection processor 1100, applies the MIP 4010 to the OCTA image 4000 to generate the MIP image 4020 (S62). The MIP image 4020 generated is stored in the memory 212 by the main controller 211.

Following that, using the blood vessel enhancement processor 1200, the ophthalmic apparatus 1 applies the multiscale Frangi filter 4030 to the MIP image 4020 to generate the first blood vessel enhanced image 4040 (S63). The first blood vessel enhanced image 4040 generated is stored in the memory 212 by the main controller 211.

By employing the image projection processor 1100, the ophthalmic apparatus 1 applies the AIP 4050 to the OCTA image 4000 to generate the AIP image 4060 (S64). The AIP image 4060 generated is stored in the memory 212 by the main controller 211.

Thereafter, using the blood vessel enhancement processor 1200, the ophthalmic apparatus 1 applies the Frangi filter 4070 of the scale corresponding to the range of the width of the capillary to the AIP image 4060, thereby generating the Frangi filtered image 4080 in which the capillary is depicted (S65). The Frangi filtered image 4080 generated is stored in the memory 212 by the main controller 211.

Furthermore, using the blood vessel enhancement processor 1200, the ophthalmic apparatus 1 applies the gamma correction 4090 configured for increasing brightness of the blood vessel image to the Frangi filtered image 4080, thereby generating the second blood vessel enhanced image 4100 (S66). The second blood vessel enhanced image 4100 generated is stored in the memory 212 by the main controller 211.

Subsequently, by the use of the image compositing processor 1400, the ophthalmic apparatus 1 generates the composite image 4120 by applying the image compositing process 4110 to the first blood vessel enhanced image 4040, the second blood vessel enhanced image 4100, and the MIP image 4020 (S67). The composite image 4120 generated is stored in the memory 212 by the main controller 211. This concludes the explanation of the present operation example (End).

The ophthalmic apparatus 1 may display any of the images acquired or generated in the operation example shown in FIGS. 10A and 10B. The ophthalmic apparatus 1 may apply a freely selected or designed process to any of the images acquired or generated in the operation example shown in FIGS. 10A and 10B. The ophthalmic apparatus 1 may transmit, to an external device, any of the images acquired or generated in the operation example shown in FIGS. 10A and 10B. The ophthalmic apparatus 1 may record, in a recording medium, any of the images acquired or generated in the operation example shown in FIGS. 10A and 10B.

The ophthalmic apparatus 1 according to the second embodiment example is configured to generate the first and second projection images from the OCTA image using two mutually different image projection methods. For example, an MIP image with excellent blood vessel depiction capabilities is generated as the first projection image, and an AIP image with low-noise is generated as the second projection image.

The ophthalmic apparatus 1 may be configured to apply the multiscale Frangi filter to the first projection image to generate the first blood vessel enhanced image in which the images of blood vessels of various thicknesses are depicted. Along with this, the ophthalmic apparatus 1 may also be configured to apply the Frangi filter of a specific scale to the second projection image to generate the Frangi filtered image in which thin blood vessel images are enhanced.

Thus, in the Frangi filtered image, thin blood vessel images are enhanced while the intensity of signals representing the thin blood vessel images is low. Accordingly, there is a possibility, in the MIP process, that noise with higher signal intensity than the thin blood vessel images may be selected. In contrast, in the AIP process, such a problem is less likely to occur since the signal intensities in the depth direction are averaged. Therefore, the thin blood vessel images in the OCTA image can be accurately depicted in the second projection image generated from the OCTA image by using the AIP process. Furthermore, the thin blood vessel images in the OCTA image are also accurately depicted in the Frangi filtered image generated from the second projection image by using the Frangi filter of the specific scale.

Further, the second embodiment example applies the gamma correction to the Frangi filtered image, thereby increasing the brightness (and contrast) of the thin blood vessel images depicted in the Frangi filtered image and therefore improving the clarity of the thin blood vessel images.

Furthermore, in the second embodiment example, the Frangi filtered image (i.e., the second blood vessel enhanced image used as the denoised image) in which the thin blood vessel images have been made clearer through the gamma correction, is composed with the first blood vessel enhanced image generated by applying the multiscale Frangi filter to the OCTA image. Accordingly, it is possible to compensate for dropout or lack of clarity of the thin blood vessel images depicted in the first blood vessel enhanced image generated using MIP, by utilizing the thin blood vessel images that are clearly depicted in the second blood vessel enhanced image.

Moreover, in the second embodiment, it is possible to generate a composite image having a texture closer to that of the original image by composing not only the second blood vessel enhanced image, which is a denoised image, but also the first projection image (e.g., an MIP image with high blood vessel depiction capability) with the first blood vessel enhanced image generated using the multiscale Frangi filter.

As described above, in some aspect examples, MIP is used as the first projection process and AIP is used as the second projection process. The combination of two mutually different projection processes is not limited to the combination of MIP and AIP. In some examples, a freely selected or designed type of image projection method with excellent blood vessel depiction capability may be used for the first projection process, and/or a freely selected or designed type of image projection method with excellent noise reduction capability may be used for the second projection process.

In some aspect examples, the processes according to the second embodiment example may be applied to a specific site of the eye fundus as a target. Since the second embodiment example is intended to eliminate dropout of thin blood vessel images, the target site may be one in which capillaries are present, and in particular, may be a site in which capillaries are densely distributed.

A non-limiting example of the target blood vessels is the RPCs. The RPCs are retinal blood vessels derived from the central retinal artery and are located in the most superficial layer of the retina. In the present example, the OCTA image acquired by the image acquisition unit 1000 is an image in which a region of the fundus Ef including the RPCs is depicted. Such an image can be acquired by applying OCT scanning to a region of the fundus Ef that includes the RPCs.

Even in cases where blood vessels other than the RPCs are targeted, the processes according to the second embodiment example and the process of acquiring the OCTA image can be performed in the same manner as described above.

### Third Embodiment Example

In the third embodiment example, a description will be given of several aspects of the processes for addressing the third problem (i.e., the problem of noise in the image of the avascular region) caused by the use of the blood vessel enhancing filter.

The third problem that the third embodiment example deals with corresponds to the phenomenon in which noise that has occurred in a region of the fundus where no blood vessels should be present is enhanced by the blood vessel enhancing filter and consequently visualized as if it were a blood vessel image. This phenomenon has been found to be particularly prominent in the FAZ.

In the third embodiment example, the blood vessel enhancement processor 1200 is configured to apply the multiscale Frangi filter to the projection image generated from the OCTA image by the image projection processor 1100, thereby generating a blood vessel enhanced image.

In some aspect examples, the projection image is an image generated using a projection method with highly effective blood vessel depiction capabilities. Such a projection image may be, for example, an MIP image generated using MIP.

In the third embodiment example, the denoising processor 1300 may be configured to apply an avascular region identifying process, which is configured for identifying an avascular region image corresponding to an avascular region of the fundus Ef of the subject's eye E, to the blood vessel enhanced image generated by the blood vessel enhancement processor 1200.

The avascular region to be identified may be a predetermined site or a site determined based on the blood vessel enhanced image. An example of the former site is the FAZ. An example of the latter site is a site corresponding to an image region in which the density of blood vessel images is equal to or less than a predetermined threshold.

The avascular region identifying process may include a freely selected or configured process. In some aspect examples, the process described in International Publication No. WO 2019/203056 may be employed. Further, in some aspect examples, any process described in the aspect examples and the operation examples described below may be employed.

In the third embodiment example, the denoising processor 1300 may be further configured to generate the denoised image by applying a masking process based on the avascular region image identified by using the avascular region identifying process to the blood vessel enhanced image generated by the blood vessel enhancement processor 1200. A non-limiting example of the masking process will be described later.

In the third embodiment example, the image compositing processor 1400 may be configured to generate the composite image by applying the image compositing process to the denoised image and the projection image. Here, the denoised image is the image generated by the denoising processor 1300 by applying the masking process based on the avascular region image to the blood vessel enhanced image, and the projection image is the image generated by the image projection processor 1100 from the OCTA image. The image compositing process may be performed using alpha blending or another image compositing method.

In some aspect examples, the denoising processor 1300 may be configured to perform, in the avascular region identifying process, the first filtering process that applies a variance filter to the blood vessel enhanced image generated by the blood vessel enhancement processor 1200.

In addition, in some aspect examples, the denoising processor 1300 may be configured to determine, in the avascular region identifying process, the first brightness threshold based on a variance filtered image generated from the blood vessel enhanced image by the first filtering process using the variance filter. This process is referred to as the first brightness threshold determining process. Furthermore, the denoising processor 1300 may be configured to generate, in the avascular region identifying process, the first mask image by applying a thresholding process with the first brightness threshold determined by the first brightness threshold determining process to the variance filtered image generated from the blood vessel enhanced image by the first filtering process using the variance filter. This process is referred to as the first thresholding process.

In some aspect examples, the denoising processor 1300 may be configured to perform, in the avascular region identifying process, the second filtering process that applies a mean filter to the projection image generated from the OCTA image by the image projection processor 1100.

In some aspect examples, the denoising processor 1300 may be configured to perform, in the avascular region identifying process, two filtering processes as the following: the first filtering process that applies the variance filter to the blood vessel enhanced image generated by the blood vessel enhancement processor 1200, and the second filtering process that applies the mean filter to the projection image generated by the image projection processor 1100.

In some aspect examples, the denoising processor 1300 may be configured to perform, in the avascular region identifying process, the following processes: the first brightness threshold determining process that determines the first brightness threshold based on the variance filtered image generated by the first filtering process; the first thresholding process that applies a thresholding process with the first brightness threshold to the variance filtered image, thereby generating the first mask image; and the second filtering process that applies the mean filter to the projection image generated by the image projection processor 1100.

Further, in some aspect examples, the denoising processor 1300 may be configured to perform, in the avascular region identifying process, the following processes: the second brightness threshold determining process that determines the second brightness threshold based on the mean filtered image generated by the second filtering process; and the second thresholding process that applies a thresholding process with the second brightness threshold to the mean filtered image, thereby generating the second mask image.

Still further, in some aspect examples, the denoising processor 1300 may be configured to perform, in the avascular region identifying process, the following processes: the process of composing the first mask image and the second mask image to generate a composite mask image; and the process of generating the avascular region image based on the composite mask image.

In addition, in some aspect examples, the denoising processor 1300 may be configured to generate a summation image of the first mask image and the second mask image as the composite mask image in the avascular region identifying process.

Moreover, in some aspect examples, the denoising processor 1300 may be configured to generate the avascular region image by applying a gaussian filter to the summation image of the first mask image and the second mask image in the avascular region identifying process.

More generally, in some aspect examples, the denoising processor 1300 may be configured to generate the avascular region image by applying a gaussian filter to the composite mask image generated by composing the first mask image and the second mask image in the avascular region identifying process.

Even more generally, in some aspect examples, the denoising processor 1300 may be configured to generate the avascular region image using a gaussian filter in the avascular region identifying process.

The third embodiment example is capable of generating a blood vessel enhanced OCTA image without the problem of noise occurring in the image of the avascular region, through a series of processes that can be realized by the configuration described above.

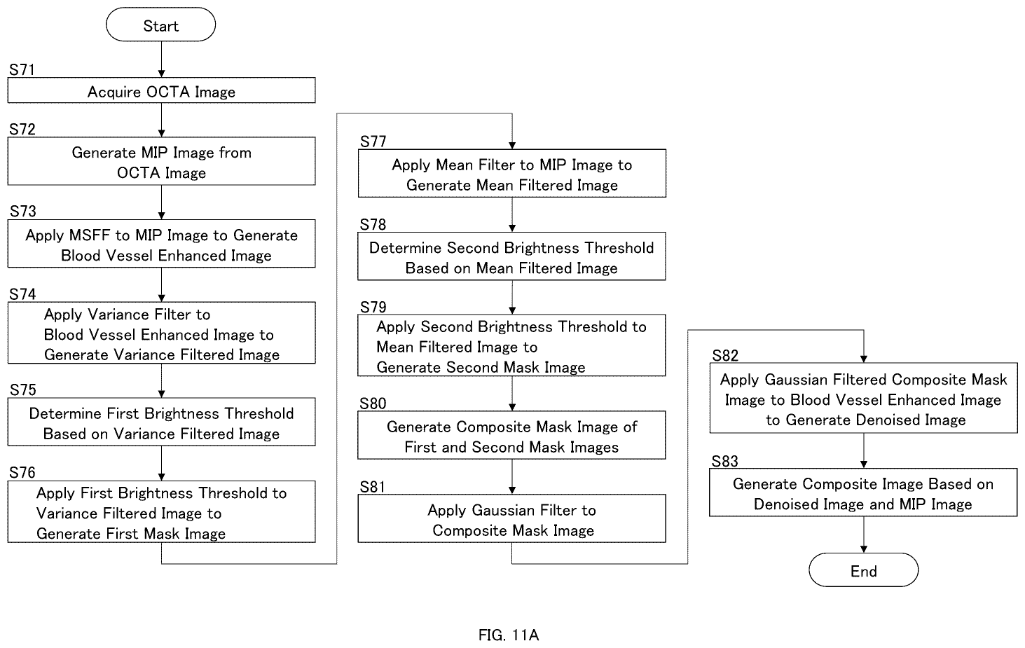

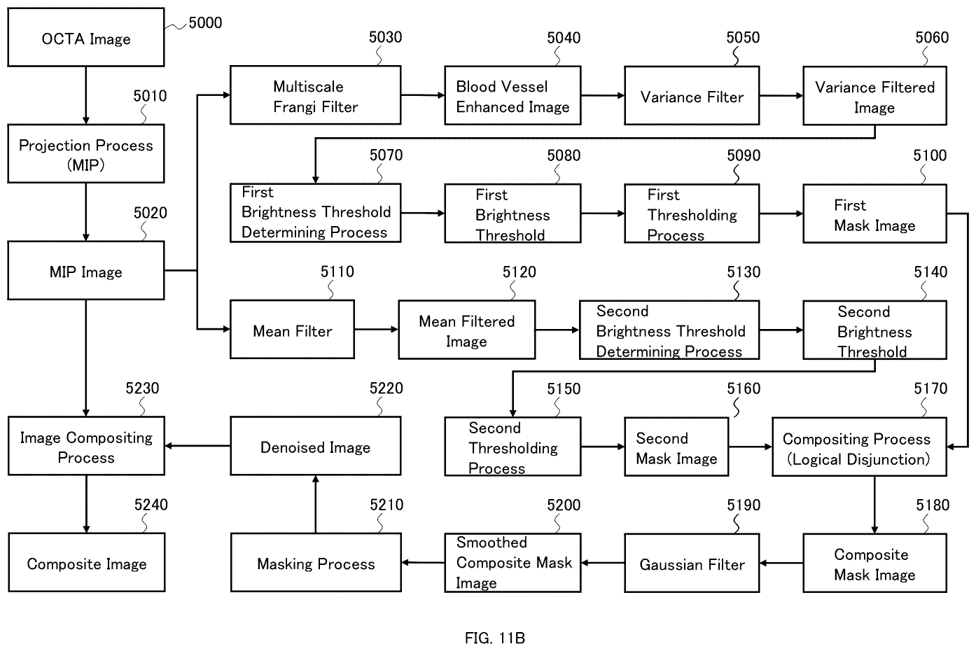

Subsequently, the operation of the ophthalmic apparatus 1 of the third embodiment example will be described with reference to FIGS. 11A and 11B.

In the operation example illustrated in FIGS. 11A and 11B, the ophthalmic apparatus 1 first acquires the OCTA image 5000 of the fundus Ef of the subject's eye E by the use of the image acquisition unit 1000 (S71). The OCTA image 5000 acquired is stored in the memory 212 by the main controller 211.

Next, the ophthalmic apparatus 1, using the image projection processor 1100, generates the MIP image 5020 by applying the MIP 5010 to the OCTA image 5000 (S72). The MIP image 5020 generated is stored in the memory 212 by the main controller 211. In the present operation example, MIP that has excellent blood vessel depiction capabilities is used. Note that any image projection method other than MIP may be used.

After the step S72, the following two process lines are performed: the processes of the steps S73 to S76 (corresponding to the reference characters 5030 to 5100 shown in FIG. 11B), and the processes of the steps S77 to S79 (corresponding to the reference characters 5110 to 5160 shown in FIG. 11B). The order in which these two process lines are performed may be freely determined. The two process lines may be performed in parallel, at least in part.

Using the blood vessel enhancement processor 1200, the ophthalmic apparatus 1 applies the multiscale Frangi filter 5030 to the MIP image 5020 to generate the blood vessel enhanced image 5040 (S73). The blood vessel enhanced image 5040 generated is stored in the memory 212 by the main controller 211.

In the blood vessel enhanced image 5040 generated using the multiscale Frangi filter 5030, images of blood vessels of various thicknesses are depicted and also an image of the FAZ is depicted. The image region in the blood vessel enhanced image 5040 that corresponds to the FAZ is referred to as an avascular region image. In the avascular region image of the blood vessel enhanced image 5040, noise appears as a result of noise signals being enhanced. In the case where a noise signal having gradient information analogous to that of blood vessels is included in the original image (e.g., the OCTA image 5000, the MIP image 5020), this noise signal is enhanced by the multiscale Frangi filter 5030 and appears as vessel-like noise within the avascular region image of the blood vessel enhanced image 5040. Such noise is enhanced even if the scale of the Frangi filter is adjusted. In order to address this type of noise, the present operation example performs a series of processes as described below.

By employing the denoising processor 1300, the ophthalmic apparatus 1 applies the variance filter 5050 to the blood vessel enhanced image 5040 to generate the variance filtered image 5060 (S74). The variance filtered image 5060 generated is stored in the memory 212 by the main controller 211.

The identification of the avascular region image by the use of the variance filter 5050 is a filtering process that utilizes the characteristic that the magnitude of the variance values in a region where a blood vessel signal is present differs from the magnitude of the variance values in a region where a blood vessel signal is absent. This avascular region image identification process is performed in order to prevent the enhancement of blood vessels from being reflected in the FAZ. The variance filter 5050 functions to identify an image region where there is no blood vessel signal in the blood vessel enhanced image 5040. In the present operation example, the avascular region image in the blood vessel enhanced image 5040 is detected. The variance filtered image 5060 generated by applying the variance filter 5050 to the blood vessel enhanced image 5040 includes the avascular region image.

In the case where the size of the variance filter 5050 is too small, not only the avascular region image but also small regions may be detected and subsequently masked in the later process. In order to selectively detect the avascular region image, the variance filter 5050 of an appropriate size is prepared. In the alternative, the size of the variance filter 5050 may be adaptively determined in accordance with the size of the image (e.g., the OCTA image 5000, the MIP image 5020, the blood vessel enhanced image 5040, etc.). As a further alternative, the size of the variance filter 5050 may be determined according to the object depicted in the image.

Next, the ophthalmic apparatus 1 performs, by the denoising processor 1300, the first brightness threshold determining process 5070 that determines the first brightness threshold 5080 based on the variance filtered image 5060 (S75). The first brightness threshold 5080 determined is stored in the memory 212 by the main controller 211.

In the variance filtered image 5060, a signal of a certain intensity (blood vessel signal) is present in a region where a blood vessel exists (referred to as a blood vessel region), whereas no blood vessel signal is present in a region where no blood vessels are located (referred to as a background region). In the first brightness threshold determining process 5070, for example, a histogram of the brightness values of the variance filtered image 5060 is generated, and based on this histogram, a threshold is determined that is used for distinguishing between a signal corresponding to a blood vessel (a region corresponding to a blood vessel) and a signal not corresponding to a blood vessel (a region not corresponding to a blood vessel). The threshold determined in this way is used as the first brightness threshold 5080.

Following this, by the use of the denoising processor 1300, the ophthalmic apparatus 1 applies the first thresholding process 5090 with the first brightness threshold 5080 to the variance filtered image 5060, thereby generating the first mask image 5100 (S76). The first mask image 5100 generated is stored in the memory 212 by the main controller 211.

The first mask image 5100 provides a mask that covers the area of the avascular region image determined by the processes of the steps S74 to S76 (corresponding to the reference characters 5050 to 5090 shown in FIG. 11B).

In some aspect examples, the processes of the steps S77 to S80 may be omitted, and the processes of the steps S81 to S83 may be performed using the first mask image 5100 generated through the processes the steps S74 to S76. In this case, advantages include simplification and shortening of the process. On the other hand, a potential disadvantage is a reduction in the robustness of the process for identifying the avascular region image. In the present operation example, the processes of the steps S77 to S80 are performed to improve the robustness of the process for identifying the avascular region image.

The ophthalmic apparatus 1 applies the mean filter 5110 to the MIP image 5020 generated in the step S72 to generate the mean filtered image 5120 (S77). The mean filtered image 5120 generated is stored in the memory 212 by the main controller 211.

The identification of the avascular region image using the mean filter 5110 is a filtering process that takes advantage of the characteristic difference between signal intensity of the blood vessel region and signal intensity of the background region. Similar to the identification of the avascular region image using the variance filter 5050, the identification of the avascular region image using the mean filter 5110 is performed to prevent blood vessel enhancement from being reflected in the FAZ. The mean filter 5110 functions to identify image regions without blood vessel signals in the blood vessel enhanced image 5040. In the present operation example, the avascular region image in the blood vessel enhanced image 5040 is detected. The avascular region image is included in the mean filtered image 5120 generated by applying the mean filter 5110 to the blood vessel enhanced image 5040.

In subsequent processing, a lower limit value of brightness is set in order to mask a region with low brightness. The lower limit value of brightness may be set in advance. Alternatively, the lower limit value of brightness may be set in accordance with the size of the image (e.g., the OCTA image 5000, the MIP image 5020, etc.). As a further alternative, the lower limit value of brightness may be set in accordance with an object depicted in the image (e.g., the OCTA image 5000, the MIP image 5020, etc.). Further, in some typical aspect examples, the size of the mean filter 5110 is set to be equal to the size of the variance filter 5050. However, the size of the variance filter 5050 and the size of the mean filter 5110 may be different from each other.

Next, the ophthalmic apparatus 1 performs, by the denoising processor 1300, the second brightness threshold determining process 5130 that determines the second brightness threshold 5140 based on the mean filtered image 5120 (S78). The second brightness threshold 5140 determined is stored in the memory 212 by the main controller 211.

In the mean filtered image 5120, a blood vessel signal of a certain intensity is present in the blood vessel region while no blood vessel signal is present in the background region. In the second brightness threshold determining process 5130, for example, a histogram of the brightness values of the mean filtered image 5120 is generated, and based on this histogram, a threshold is determined that is used for distinguishing between a blood vessel signal and a background signal (between the blood vessel region and the background region). This threshold is used as the second brightness threshold 5140.

Following this, by the use of the denoising processor 1300, the ophthalmic apparatus 1 applies the second thresholding process 5150 with the second brightness threshold 5140 to the mean filtered image 5120, thereby generating the second mask image 5160 (S79). The second mask image 5160 generated is stored in the memory 212 by the main controller 211.

The second mask image 5160 provides a mask that covers the area of the avascular region image determined by the processes of the steps S77 to S79 (corresponding to the reference characters 5110 to 5150 shown in FIG. 11B).

In this manner, the present operation example obtains the following two mask images: the first mask image 5100 that represents the area of the avascular region image determined by the processes of the steps S74 to S76 (corresponding to the reference characters 5050 to 5090 shown in FIG. 11B); and the second mask image 5160 that represents the area of the avascular region image determined by the processes of the steps S77 to S79 (corresponding to the reference characters 5110 to 5150 shown in FIG. 11B). Since the two mask images 5110 and 5160 are generated through mutually different processes, the area of the avascular region image represented by the first mask image 5100 and the area of the avascular region image represented by the second mask image 5160 typically differ from each other. The present operation example is configured to use both the mask images 5100 and 5160, thereby improving the robustness of the process of identifying the avascular region image.

After having completed the two process lines, the steps S73 to S76 (corresponding to the reference characters 5030 to 5100 shown in FIG. 11B) and the steps S77 to S79 (corresponding to the reference characters 5110 to 5160 shown in FIG. 11B), the ophthalmic apparatus 1, by using the denoising processor 1300, generates the composite mask image 5180 by applying the compositing process 5170 to the first mask image 5100 and the second mask image 5160 (S80). The composite mask image 5180 generated is stored in the memory 212 by the main controller 211.

In the present operation example, a logical disjunction (OR) operation is performed on the first mask image 5100 and the second mask image 5160 in the compositing process 5170. As a result, an image representing the union of the first mask image 5100 and the second mask image 5160 (summation image) is generated as the composite mask image 5180. This yields the composite mask image 5180 that indicates the region determined to be the avascular region image by any one or both of the variance filter 5050 and the mean filter 5110.

In some aspect examples, another operation may be performed in the compositing process 5170. In the compositing process 5170 of some examples, a logical conjunction (AND) operation may be performed on the first mask image 5100 and the second mask image 5160 to generate an image representing the intersection of the first mask image 5100 and the second mask image 5160 (intersection image) as the composite mask image 5180. In this case, obtained is the composite mask image 5180 that indicates the region determined to be the avascular region image by both the variance filter 5050 and the mean filter 5110. Note that a plurality of operations may be selectively performed in the compositing process 5170.

Next, the ophthalmic apparatus 1 applies, by the denoising processor 1300, the gaussian filter 5190 to the composite mask image 5180 (S81).

The contour (boundary) of the composite mask image 5180 generated using the thresholding process (5090 and 5150) is sharp, but the boundary can be smoothed (blurred) by the gaussian filter 5190. The composite mask image 5180 to which the gaussian filter 5190 has been applied is referred to as the smoothed composite mask image 5200. The smoothed composite mask image 5200 is stored in the memory 212 by the main controller 211. It should be noted that the application of the gaussian filter 5190 may be optional.

Subsequently, by using the denoising processor 1300, the ophthalmic apparatus 1 performs the masking process 5210 that applies the smoothed composite mask image 5200 to the blood vessel enhanced image 5040, thereby generating the denoised image 5220 (S82). The denoised image 5220 generated is stored in the memory 212 by the main controller 211. The denoised image 5220 corresponds to the blood vessel enhanced image 5040 with a mask applied to the region corresponding to the smoothed composite mask image 5200.

Thereafter, using the image compositing processor 1400, the ophthalmic apparatus 1 generates the composite image 5240 by applying the image compositing process 5230 to the denoised image 5220 and the MIP image 5020 (S83).

In the image compositing process 5230, for the mask region based on the smoothed composite mask image 5200, the corresponding region of the MIP image 5020 is adopted. In addition, for the region other than the mask region, the blood vessel enhanced image 5040 and the MIP image 5020 are composed. The composite image 5240 generated is stored in the memory 212 by the main controller 211. This concludes the explanation of the present operation example (End).

The ophthalmic apparatus 1 may display any of the images acquired or generated in the operation example shown in FIGS. 11A and 11B. The ophthalmic apparatus 1 may apply a freely selected or designed process to any of the images acquired or generated in the operation example shown in FIGS. 11A and 11B. The ophthalmic apparatus 1 may transmit, to an external device, any of the images acquired or generated in the operation example shown in FIGS. 11A and 11B. The ophthalmic apparatus 1 may record, in a recording medium, any of the images acquired or generated in the operation example shown in FIGS. 11A and 11B.

The ophthalmic apparatus 1 according to the third embodiment example is configured to identify an avascular region image from the OCT image to be processed, and to exclude the identified avascular region image from the area to which the blood vessel enhancement with the multiscale Frangi filter is applied. In other words, the ophthalmic apparatus 1 according to the third embodiment example is configured to apply the blood vessel enhancement with the multiscale Frangi filter only to the region excluding the avascular region image. This prevents noise signals present in the avascular region image from being enhanced by the multiscale Frangi filter, and resolves the third problem (i.e., the problem of noise in the image of the avascular region). On the other hand, for areas other than the avascular region image, the blood vessels can be enhanced by using the multiscale Frangi filter. Accordingly, in regions where blood vessels should be enhanced, the blood vessel enhancement effect of the multiscale Frangi filter can be utilized, while in regions where blood vessels should not be enhanced, the blood vessel enhancement effect of the multiscale Frangi filter can be excluded.

Furthermore, in the third embodiment example, as described above, various techniques are employed to identify the avascular region image. For example, one of the remarkable effects achieved by the third embodiment example is that it improves robustness by using both the variance filter and the mean filter. More specifically, by using both the variance filter and the mean filter, for example, a region that has been mistakenly identified as avascular despite the fact that it actually contains blood vessels by the process with the variance filter can be eliminated by using the mean filter.

Moreover, according to the third embodiment example, it is possible to generate a composite image that has a texture close to that of the original image by composing a projection image with the denoised image generated from the first blood vessel enhanced image.

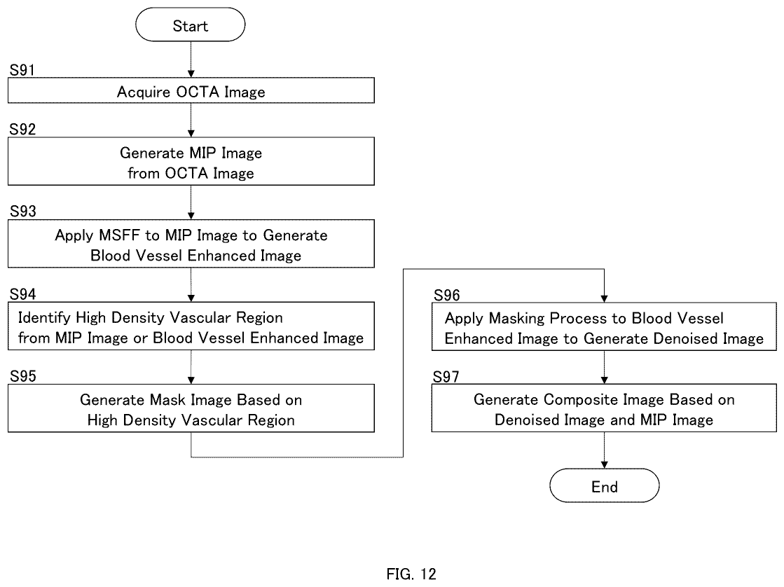

Another operation example of the ophthalmic apparatus 1 is shown in FIG. 12. In the present operation example, instead of performing a masking process based on the avascular region image identified using the above-described filtering processes (i.e., using the variance filter and the mean filter), a masking process is performed based on a region with a relatively high vascular density (referred to as a high density vascular region), thereby addressing the problem of noise in the image of the avascular region.

In the operation example shown in FIG. 12, similar to the steps S71 to S73 of FIG. 11A, the ophthalmic apparatus 1 acquires an OCTA image of the fundus Ef of the subject's eye E (S91), generates an MIP image by applying MIP to the OCTA image (S92), and applies a multiscale Frangi filter to the MIP image to generate a blood vessel enhanced image (S93). The blood vessel enhanced image generated is stored in the memory 212 by the main controller 211.

Next, using the denoising processor 1300, the ophthalmic apparatus 1 applies a process configured for identifying a high density vascular region of the fundus Ef (referred to as a high density vascular region identifying process), to the MIP image generated in the step S92 or to the blood vessel enhanced image generated in the step S93 (S94). The image corresponding to the high density vascular region of the fundus Ef identified from the MIP image or the blood vessel enhanced image is stored in the memory 212 by the main controller 211. In the alternative, the position information, in the MIP image or the blood vessel enhanced image, of the image of the identified high density vascular region, is stored in the memory 212 by the main controller 211.

The high density vascular region identifying process may be a freely selected or determined process. For example, in some aspect examples, the high density vascular region identifying process may include binarization, segmentation, variance filtering, or other types of processes.

Next, by the use of the denoising processor 1300, the ophthalmic apparatus 1 generates a mask image based on the image of the high density vascular region identified in the step S94 (S95). The mask image generated is stored in the memory 212 by the main controller 211.

The process of generating the mask image from the image of the high density vascular region may be a freely selected or determined process. For example, in view of the assumption that signals present in the vicinity of (or surrounding) signals (reliable signals) in a high density vascular region can also be identified to be blood vessel signals corresponding to blood vessels, the denoising processor 1300 may be configured to generate a mask image by expanding the image of the high density vascular region. The region outside the region resulting from the expansion of the image of the high density vascular region may be considered as the avascular region described above.

Then, using the denoising processor 1300, the ophthalmic apparatus 1 applies the masking process with the mask image generated in the step S95 to the blood vessel enhanced image generated in the step S93, thereby generating a denoised image (S96). The denoised image generated is stored in the memory 212 by the main controller 211.

In the masking process of step S96, the multiscale Frangi filter-based blood vessel enhancement effect is applied to the expanded region of the image of the high density vascular region in the blood vessel enhanced image, while the multiscale Frangi filter-based blood vessel enhancement effect is not applied to the region outside the expanded region (i.e., to the avascular region). Accordingly, noise in the avascular region is not enhanced. The denoised image generated in the step S96 is an image in which only the blood vessels in the expanded region of the high density vascular region are enhanced, and the avascular region is masked.

Subsequently, by using the image compositing processor 1400, the ophthalmic apparatus 1 generates a composite image by applying the image compositing process to the denoised image generated in the step S96 and the MIP image generated in the step S92 (S97). The composite image generated is stored in the memory 212 by the main controller 211. This concludes the explanation of the present operation example (End).

The ophthalmic apparatus 1 may display any of the images acquired or generated in the operation example shown in FIG. 12. The ophthalmic apparatus 1 may apply a freely selected or designed process to any of the images acquired or generated in the operation example shown in FIG. 12. The ophthalmic apparatus 1 may transmit, to an external device, any of the images acquired or generated in the operation example shown in FIG. 12. The ophthalmic apparatus 1 may record, in a recording medium, any of the images acquired or generated in the operation example shown in FIG. 12.

Similar to the operation example shown in FIGS. 11A and 11B, the operation example illustrated in FIG. 12 can also eliminate or reduce the problem of noise in the image of the avascular region. It is also possible to generate a composite image with a texture closer to that of the original image.

In the operation example of FIG. 12, a smoothing process may be performed to smooth (blur) the contour (boundary) of the mask image using the gaussian filter.

The robustness of the processes according to the third embodiment example can be improved by performing the following processes in combination: any one or both of the two process lines in the operation example shown in FIGS. 11A and 11B (i.e., the process using a variance filter and the process using a mean filter; and the process on the basis of the high density vascular region in the operation example shown in FIG. 12. The processes that can be adopted for this purpose are not limited to the three types of processes illustrated in the operation examples of FIGS. 11A, 11B, and 12. Improvement in robustness can be achieved by employing a process of the type other than them.

### Other Embodiment Examples

In the foregoing, various embodiment examples, aspects, and examples relating to the ophthalmic apparatus have been described. It will be understood by those skilled in the art that the present disclosure also provides embodiment examples, aspects, and examples in categories other than ophthalmic apparatuses.

A non-limiting example is the “method of processing an ophthalmic image” according to the thirty-eighth aspect example described above, and further, a method implemented by combining any of the matters or items freely selected from the various embodiment examples, aspects, and examples relating to the aforementioned ophthalmic apparatus with the thirty-eighth aspect example.

Another non-limiting example is the “method of controlling an ophthalmic apparatus” according to the thirty-ninth aspect example described above, and further, a method implemented by combining any of the matters or items freely selected from the various embodiment examples, aspects, and examples relating to the aforementioned ophthalmic apparatus with the thirty-ninth aspect example.

Still another non-limiting example is a program configured to cause a computer to execute each step in the “method of processing an ophthalmic image” according to the thirty-eighth aspect example described above, and further, a program configured to cause a computer to execute each step in a method implemented by combining any of the matters or items freely selected from the various embodiment examples, aspects, and examples relating to the aforementioned ophthalmic apparatus with the thirty-eighth aspect example.

Still another non-limiting example is a program configured to cause a computer to execute each step in the “method of controlling an ophthalmic apparatus” according to the thirty-ninth aspect example described above, and further, a program configured to cause a computer to execute each step in a method implemented by combining any of the matters or items freely selected from the various embodiment examples, aspects, and examples relating to the aforementioned ophthalmic apparatus with the thirty-ninth aspect example.

Further, another non-limiting example is a computer-readable non-transitory recording medium storing the program configured to cause a computer to execute each step in the “method of processing an ophthalmic image” according to the thirty-eighth aspect example described above, and further, a computer-readable non-transitory recording medium storing the program configured to cause a computer to execute each step in the method implemented by combining any of the matters or items freely selected from the various embodiment examples, aspects, and examples relating to the aforementioned ophthalmic apparatus with the thirty-eighth aspect example.

Further still, another non-limiting example is a computer-readable non-transitory recording medium storing the program configured to cause a computer to execute each step in the “method of controlling an ophthalmic apparatus” according to the thirty-ninth aspect example described above, and further, a computer-readable non-transitory recording medium storing the program configured to cause a computer to execute each step in the method implemented by combining any of the matters or items freely selected from the various embodiment examples, aspects, and examples relating to the aforementioned ophthalmic apparatus with the thirty-ninth aspect example.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

While certain embodiments have been described, these embodiments have been presented by way of example only, and are not intended to limit the scope of the inventions. Indeed, the novel embodiments described herein may be embodied in a variety of other forms; furthermore, various omissions, substitutions, additions and changes in the form of the embodiments described herein may be made without departing from the spirit of the inventions. The accompanying claims and their equivalents are intended to cover such forms or modifications as would fall within the scope and spirit of the inventions.

1. An ophthalmic apparatus comprising:
an image acquisition unit including a scanner and configured to acquire an optical coherence tomography angiography image of a fundus of a subject's eye;
an image projection processor configured to apply a projection process to the optical coherence tomography angiography image to generate a projection image;
a blood vessel enhancement processor configured to apply a blood vessel enhancing filter that is configured to enhance a blood vessel image to the projection image to generate a blood vessel enhanced image;
a denoising processor configured to apply a denoising process to the projection image to generate a denoised image; and
an image compositing processor configured to apply an image compositing process to the projection image, the blood vessel enhanced image, and the denoised image to generate a composite image.

2. The ophthalmic apparatus of claim 1, wherein the blood vessel enhancing filter includes a multiscale Frangi filter.

3. The ophthalmic apparatus of claim 1, wherein the denoising processor is configured to be able to perform a plurality of processes of mutually different types in the denoising process.

4. The ophthalmic apparatus of claim 3, wherein
the denoising processor is configured to select at least one process from the plurality of processes based on a field of view of the optical coherence tomography angiography image, and generate the denoised image by applying the at least one process to the projection image, and
the image compositing processor is configured to generate the composite image by applying the image compositing process to the denoised image generated by applying the at least one process to the projection image, the projection image, and the blood vessel enhanced image.

5. The ophthalmic apparatus of claim 3, wherein
the denoising processor is configured to select at least one process from the plurality of processes based on at least one of an eye fixation position and a scan area that are used for generating the optical coherence tomography angiography image, and generate the denoised image by applying the at least one process to the projection image, and
the image compositing processor is configured to generate the composite image by applying the image compositing process to the denoised image generated by applying the at least one process to the projection image, the projection image, and the blood vessel enhanced image.

6. The ophthalmic apparatus of claim 1, wherein
the denoising processor is configured to apply a blood vessel image extracting process that extracts a blood vessel image with a width belonging to a first range to the projection image, and apply an erosion process to an image generated through the blood vessel image extracting process to generate an eroded image in which a reduced blood vessel image corresponding to the extracted blood vessel image with a width reduced by the erosion process is depicted, and
the blood vessel enhancement processor is configured to apply a multiscale Frangi filter to the projection image to generate a first blood vessel enhanced image, and apply a Frangi filter of a scale corresponding to a second range smaller than the first range to the projection image and further apply gamma correction that increases brightness of a blood vessel image to an image generated by this Frangi filter to generate a second blood vessel enhanced image,
the denoising processor is configured to generate the denoised image based on the eroded image, the first blood vessel enhanced image, and the second blood vessel enhanced image, and
the image compositing processor is configured to generate the composite image by applying the image compositing process to the projection image, the first blood vessel enhanced image, and the denoised image generated from the eroded image, the first blood vessel enhanced image, and the second blood vessel enhanced image.

7. The ophthalmic apparatus of claim 6, wherein
the denoising processor is configured to identify a first sub-image of the first blood vessel enhanced image that corresponds to the reduced blood vessel image in the eroded image, identify a second sub-image of the second blood vessel enhanced image that corresponds to the reduced blood vessel image in the eroded image, and generate the denoised image by selecting a higher brightness value between a brightness value of a pixel of the first sub-image and a brightness value of a corresponding pixel of the second sub-image, and
the image compositing processor is configured to generate the composite image by applying the image compositing process to the projection image, the first blood vessel enhanced image, and the denoised image generated from the first sub-image and the second sub-image.

8. The ophthalmic apparatus of claim 7, wherein the denoising processor is configured to determine the first sub-image by applying a masking process based on the reduced blood vessel image in the eroded image to the first blood vessel enhanced image, and determine the second sub-image by applying the masking process to the second blood vessel enhanced image.

9. The ophthalmic apparatus of claim 1, wherein
the denoising processor is configured to apply a blood vessel image extracting process that extracts a blood vessel image with a width belonging to a first range to the projection image, analyze the blood vessel image extracted by the blood vessel image extracting process to determine a centerline of the blood vessel image, determine a brightness profile with respect to distance from the centerline, and apply a process based on the brightness profile to an image generated through the blood vessel image extracting process to generate a processed image in which a reduced blood vessel image corresponding to the extracted blood vessel image with a reduced width is depicted,
the blood vessel enhancement processor is configured to apply a multiscale Frangi filter to the projection image to generate a first blood vessel enhanced image, and apply a Frangi filter of a scale corresponding to a second range smaller than the first range to the projection image and further apply gamma correction that increases brightness of a blood vessel image to an image generated by this Frangi filter to generate a second blood vessel enhanced image,
the denoising processor is configured to generate the denoised image based on the processed image, the first blood vessel enhanced image, and the second blood vessel enhanced image, and
the image compositing processor is configured to generate the composite image by applying the image compositing process to the projection image, the first blood vessel enhanced image, and the denoised image generated from the processed image, the first blood vessel enhanced image, and the second blood vessel enhanced image.

10. The ophthalmic apparatus of claim 9, wherein
the denoising processor is configured to identify a first sub-image of the first blood vessel enhanced image that corresponds to the reduced blood vessel image in the processed image, identify a second sub-image of the second blood vessel enhanced image that corresponds to the reduced blood vessel image in the processed image, and generate the denoised image by selecting a higher brightness value between a brightness value of a pixel of the first sub-image and a brightness value of a corresponding pixel of the second sub-image, and
the image compositing processor is configured to generate the composite image by applying the image compositing process to the projection image, the first blood vessel enhanced image, and the denoised image generated from the first sub-image and the second sub-image.

11. The ophthalmic apparatus of claim 1, wherein
the image projection processor is configured to apply a first projection process to the optical coherence tomography angiography image to generate a first projection image, and apply a second projection process that is different from the first projection process to the optical coherence tomography angiography image to generate a second projection image,
the blood vessel enhancement processor is configured to apply a multiscale Frangi filter to the first projection image to generate a first blood vessel enhanced image, and apply a Frangi filter of a scale corresponding to a width of a capillary to the second projection image and further apply gamma correction that increases brightness of a blood vessel image to an image generated by this Frangi filter to generate a second blood vessel enhanced image as the denoised image, and
the image compositing processor is configured to generate the composite image by applying the image compositing process to the first projection image, the first blood vessel enhanced image, and the second blood vessel enhanced image.

12. The ophthalmic apparatus of claim 11, wherein
the first projection process is maximum intensity projection, and
the second projection process is average intensity projection.

13. The ophthalmic apparatus of claim 1, wherein
the blood vessel enhancement processor is configured to apply a multiscale Frangi filter to the projection image to generate a blood vessel enhanced image,
the denoising processor is configured to apply an avascular region identifying process that identifies an avascular region image corresponding to an avascular region of the fundus to the blood vessel enhanced image, and generate the denoised image by applying a masking process based on the avascular region image identified by the avascular region identifying process to the blood vessel enhanced image, and
the image compositing processor is configured to generate the composite image by applying the image compositing process to the projection image and the denoised image generated by applying the masking process based on the avascular region image to the blood vessel enhanced image.

14. The ophthalmic apparatus of claim 13, wherein the denoising processor is configured to perform, in the avascular region identifying process, a first filtering process that applies a variance filter to the blood vessel enhanced image.

15. The ophthalmic apparatus of claim 14, wherein the denoising processor is configured to perform, in the avascular region identifying process, a first brightness threshold determining process that determines a first brightness threshold based on a variance filtered image generated by the first filtering process, and a first thresholding process that applies a thresholding process with the first brightness threshold to the variance filtered image to generate a first mask image.

16. The ophthalmic apparatus of claim 15, wherein the denoising processor is configured to perform, in the avascular region identifying process, a second filtering process that applies a mean filter to the projection image.

17. The ophthalmic apparatus of claim 16, wherein the denoising processor is configured to perform, in the avascular region identifying process, a second brightness threshold determining process that determines a second brightness threshold based on a mean filtered image generated by the second filtering process, and a second thresholding process that applies a thresholding process with the second brightness threshold to the mean filtered image to generate a second mask image.

18. The ophthalmic apparatus of claim 17, wherein the denoising processor is configured to compose the first mask image and the second mask image to generate a composite mask image and generate the avascular region image based on the composite mask image in the avascular region identifying process.

19. The ophthalmic apparatus of claim 18, wherein the denoising processor is configured to generate a summation image of the first mask image and the second mask image as the composite mask image in the avascular region identifying process.

20. The ophthalmic apparatus of claim 19, wherein the denoising processor is configured to generate the avascular region image by applying a gaussian filter to the summation image in the avascular region identifying process.

21. The ophthalmic apparatus of claim 1, wherein
the blood vessel enhancement processor is configured to apply a multiscale Frangi filter to the projection image to generate a blood vessel enhanced image,
the denoising processor is configured to apply a high density vascular region identifying process that identifies a high density vascular region image corresponding to a high density vascular region of the fundus to the projection image or the blood vessel enhanced image, and generate the denoised image by applying a masking process based on the high density vascular region image identified by the high density vascular region identifying process to the blood vessel enhanced image, and
the image compositing processor is configured to generate the composite image by applying the image compositing process to the projection image and the denoised image generated by applying the masking process based on the high density vascular region image to the blood vessel enhanced image.

22. A method of processing an optical coherence tomography angiography image of a fundus of a subject's eye by using a computer including a processor, memory, and a data input interface, the method comprising:
an inputting process step performed by the data input interface to input the optical coherence tomography angiography image into the computer;
a storing process step performed by the memory to store the optical coherence tomography angiography image;
an image projection process step performed by the processor to apply a projection process to the optical coherence tomography angiography image stored in the memory to generate a projection image;
a blood vessel enhancement process step performed by the processor to apply a blood vessel enhancing filter that is configured to enhance a blood vessel image to the projection image to generate a blood vessel enhanced image;
a denoising process step performed by the processor to apply a denoising process to the projection image to generate a denoised image; and
an image compositing process step performed by the processor to apply an image compositing process to the projection image, the blood vessel enhanced image, and the denoised image to generate a composite image.

23. A method of controlling an ophthalmic apparatus including a processor, memory, and an image acquisition device, the method comprising:
an image acquisition control step of controlling the image acquisition device to acquire an optical coherence tomography angiography image of a fundus of a subject's eye;
a storing control step of controlling the memory to store the optical coherence tomography angiography image;
an image projection control step of controlling the processor to apply a projection process to the optical coherence tomography angiography image stored in the memory to generate a projection image;
a blood vessel enhancement control step of controlling the processor to apply a blood vessel enhancing filter that is configured to enhance a blood vessel image to the projection image to generate a blood vessel enhanced image;
a denoising control step of controlling the processor to apply a denoising process to the projection image to generate a denoised image; and
an image compositing control step of controlling the processor to apply an image compositing process to the projection image, the blood vessel enhanced image, and the denoised image to generate a composite image.
